(19) United States
(12) Patent Application Publication
(10) Pub. No.: US 2018/0020908 A1
(43) Pub. Date: Jan. 25, 2018

(54) SYSTEMS AND METHODS FOR DETERMINING RETINAL GANGLION CELL POPULATIONS AND ASSOCIATED TREATMENTS

(71) Applicant: The Regents of the University of California, Oakland, CA (US)

(72) Inventors: Felipe A. Medeiros, San Diego, CA (US); Robert N. Weinreb, Rancho Santa Fe, CA (US); Linda M. Zangwill, La Jolla, CA (US)

(21) Appl. No.: 15/643,402

(22) Filed: Jul. 6, 2017

Related U.S. Application Data
(62) Division of application No. 14/380,065, filed on Aug. 20, 2014, now abandoned.
(60) Provisional application No. 61/601,523, filed on Feb. 21, 2012.

(51) Int. Cl. A61B 3/00; G01N 33/68; A61B 3/10

(52) U.S. Cl. CPC A61B 3/0025; A61B 3/1005; A61B 3/102; G01N 33/6893; G01N 2800/168; G01N 2800/56

(57) ABSTRACT

A new combined index of structure and function (CSFI) for staging and detecting glaucomatous damage is provided. An observational study including 333 glaucomatous eyes (295 with perimetric glaucoma and 38 with preperimetric glaucoma) and 330 eyes of healthy subjects is described. All eyes were tested with standard automated perimetry (SAP) and spectral domain optical coherence tomography (SDOCT) within 6 months. Estimates of the number of retinal ganglion cells (RGC) were obtained from SAP and SDOCT and a weighted averaging scheme was used to obtain a final estimate of the number of RGCs for each eye. The CSFI was calculated as the percent loss of RGCs obtained by subtracting estimated from expected RGC numbers. The performance of the CSFI for discriminating glaucoma from normal eyes and the different stages of disease was evaluated by receiver operating characteristic (ROC) curves. The mean CSFI, representing the mean estimated percent loss of RGCs, was 41% and 17% in the perimetric and pre-perimetric groups, respectively (P<0.001). They were both significantly higher than the mean CSFI in the normal group (P<0.001). The CSFI had larger ROC curve areas than isolated indexes of structure and function for detecting perimetric and preperimetric glaucoma and differentiating among early, moderate and advanced stages of visual field loss. An index combining structure and function performed better than isolated structural and functional measures for detection of perimetric and preperimetric glaucoma as well as for discriminating different stages of the disease.

### RELATED APPLICATIONS

This application is a divisional of U.S. application Ser. No. 14/380,065, filed on Aug. 20, 2014, which is a 371 application of PCT/US2013/026962, filed on Feb. 20, 2013, and claims priority from U.S. Application No. 61/601,523, filed Feb. 21, 2012, and entitled Systems and Methods for Determining Retinal Ganglion Cell Populations and Associated Treatments. The disclosures of the above-identified applications are incorporated by reference in their entirety.

### STATEMENT REGARDING FEDERALLY SPONSORED R&D

This invention was made with government support under EY011008 and EY021818 awarded by the National Institutes of Health. The government has certain rights in the invention.

### BACKGROUND OF THE INVENTION

### Introduction

Glaucoma is an optic neuropathy characterized by progressive neuroretinal rim thinning, excavation and loss of the retinal nerve fiber layer.¹ These structural changes are usually accompanied by functional losses, which may ultimately result in a significant decrease in vision-related quality of life. Staging the severity of glaucomatous damage is an essential component in guiding management decisions and providing prognostic information. Patients with severe damage may be at an increased risk for developing functional impairment and, therefore, may require more aggressive treatment than those with mild or moderate damage. Additionally, staging systems may be used to monitor disease progression over time and also to evaluate treatment efficacy. Although both the characteristic structural and functional changes seen in the disease are ultimately related to the pathological loss of retinal ganglion cell (RGC) somas and axons, the measurements of structural and functional change are somewhat variable and have an imperfect relationship to one another, both for recognizing damage and for detecting disease progression over time. Standard automated perimetry (SAP) remains the usual method for monitoring functional changes in the disease. However, patients may present structural changes in the optic nerve or retinal nerve fiber layer (RNFL) before changes are detected with SAP.²⁻¹⁰ On the other hand, several patients show evidence of functional deterioration without measurable changes in currently available structural tests.^{5,6,11}

The most common test used to stage glaucoma severity is clinical standard automated perimetry (SAP). Visual field defects on SAP have been shown to be associated with retinal ganglion cell (RGC) loss both in experimental and clinical glaucoma.² Additionally, SAP defects are related to measures of functional impairment in the disease and, therefore, may be used to gauge the impact of the disease on quality of vision. However, experimental studies have shown that as many as 40-50% of RGCs may need to be lost before the decrease in threshold sensitivity exceeds normal variability and reaches statistical significance.²⁴ In fact, qualitative and quantitative analyses of the optic nerve and retinal nerve fiber layer (RNFL) have shown that significant structural changes are present in many patients before detectable changes in SAP⁵⁻¹³

Although many different staging schemes using SAP have been proposed, it is clear that a classification system that only considers SAP abnormalities may result in gross underestimation of the amount of damage in early disease. On the other hand, the utility of structural measurements in moderate and advanced stages of the disease has been questioned.¹⁴⁻¹⁸ There is evidence that RNFL and optic disc assessment by imaging technologies may not provide adequate sensitivity to follow patients who present with severe glaucomatous damage. In this situation, SAP losses are still the best method to quantify the impact of the disease and monitor its progression.

The apparent disagreement between structural and functional measurements of the disease seem to be largely derived from the different algorithms and measurement scales as well as the different variability characteristics of the tests commonly used to assess structural and functional losses. In fact, Harwerth and colleagues² demonstrated that structural and functional tests are in agreement as long as one uses appropriate measurement scales for neural and sensitivity losses and considers factors such as the effect of aging and eccentricity on estimates of neural losses. In a series of investigations, they demonstrated that estimates of RGC losses obtained from clinical perimetry agreed closely with estimates of RGC losses obtained from RNFL assessment by optical coherence tomography.² The results of their model provided a common domain for expressing results of structural and functional tests, e.g., the estimates of RGC losses, opening the possibility of combining these different tests to improve the reliability and accuracy of estimates of the amount of neural losses and develop a combined staging system for glaucoma severity.

### SUMMARY OF THE INVENTION

Certain embodiments contemplate a system configured to determine an index estimating a number of retinal ganglion cells (RGC) in an eye, comprising: a structure feature module configured to receive a plurality of structural feature data and to determine a structural feature estimate; a functional feature module configured to receive a plurality of functional feature data and to determine a functional feature estimate; and an index determination module configured to determine a weighted combination of the structural feature estimate and the functional feature estimate. In some of the foregoing embodiments, the plurality of functional feature data comprises standard automated perimetry data. In some of the foregoing embodiments, the plurality of structural feature data comprises optical coherence tomography data, such as spectral domain optical coherence tomography data. In some of the foregoing embodiments, the plurality of structural feature data comprises estimating the number of RGC axons from RNFL thickness measurements obtained by optical coherence tomography. In some of the foregoing embodiments, the functional feature module applies at least the following equations: m=[0.054*(ec*1.32)]+0.9; b=[−1.5*(ec*1.32)]−14.8; gc={[(s−1)−b]/m}+4.7; SAPrgc=Σ10̂(gc*0.1), wherein ec comprises the eccentricity and s comprises the sensitivity from standard automated perimetry data In some of the foregoing embodiments, the structural feature module applies at least the following equations: d=(−0.007*age)+1.4; c=(−0.26*MD)+0.12; a=average RNFL thickness*10870*d; OCTrgc=10̂[(log(a)*10−c)*0.1], wherein age is the age of the patient and MD comprises a mean deviation. In some of the foregoing embodiments, the index determination module applies at least the following formula: wrgc=(1+MD/30)*OCTrgc+(−MD/30)*SAPrgc, wherein wrgc comprises at least a portion of the index. Some embodiments further comprise a regression module, the regression module configured to relate the index to age and optic disc area in a population. In some of the foregoing embodiments, the system comprises a device selected from the group consisting of a wired device, a wireless device, a plug-in device, a computer, an external input device and a combination of any of the foregoing devices. In some of the foregoing embodiments, said system comprises manual, auditory, or visual input sources.

Certain embodiments contemplate a non-transitory computer-readable medium comprising instructions configured to cause a processor to perform at least the following: receiving a plurality of structural feature data; determining a structural feature estimate; receiving a plurality of functional feature data; determining a functional feature estimate; and determining an index based on a weighted combination of the structural feature estimate and the functional feature estimate. In some of the foregoing embodiments, the plurality of functional feature data comprises standard automated perimetry data. In some of the foregoing embodiments, the plurality of structural feature data comprises optical coherence tomography data, such as spectral domain optical coherence tomography data. In some of the foregoing embodiments, the plurality of structural feature data comprises estimating the number of RGC axons from RNFL thickness measurements obtained by optical coherence tomography. In some of the foregoing embodiments, determining a functional feature estimate comprises applying at least the following equations: m=[0.054*(ec*1.32)]+0.9; b=[−1.5*(ec*1.32)]−14.8; gc={[(s−1)−b]/m}+4.7; SAPrgc=Σ10̂(gc*0.1), wherein ec comprises the eccentricity and s comprises the sensitivity. In some of the foregoing embodiments, determining a functional feature estimate comprises applying at least the following equations: d=(−0.007*age)+1.4; c=(−0.26*MD)+0.12; a=average RNFL thickness*10870*d; OCTrgc=10̂[(log(a)*10−c)*0.1], wherein age is the age of the patient and MD comprises a mean deviation. In some of the foregoing embodiments, determining an index comprises applying at least the following formula: wrgc=(1+MD/30)*OCTrgc+(−MD/30)*SAPrgc, wherein wrgc comprises at least a portion of the index. In some of the foregoing embodiments the instructions are further configured to cause a processor to relate the index to age and optic disc area in a population. In some of the foregoing embodiments, the non-transitory computer-readable medium comprises a computer-readable storage medium. In some of the foregoing embodiments, the non-transitory computer readable medium is configured to receive data from a device selected from the group consisting of a wired device, a wireless device, a plug-in device, a computer any combination of the foregoing devices or from a source selected from the group consisting of an external input, a manual source, an auditory source, and a visual source.

Certain embodiments contemplate a method for detecting glaucoma or assessing the progression of glaucoma, comprising: receiving a plurality of structural feature data at a computer; determining a structural feature estimate at a computer; receiving a plurality of functional feature data at a computer; determining a functional feature estimate at a computer; and determining an index based on a weighted combination of the structural feature estimate and the functional feature estimate at a computer. In some of the foregoing embodiments, the plurality of functional feature data comprises standard automated perimetry data. In some of the foregoing embodiments, the plurality of structural feature data comprises optical coherence tomography data, such as spectral domain optical coherence tomography data. In some of the foregoing embodiments, the plurality of structural feature data comprises estimating the number of RGC axons from RNFL thickness measurements obtained by optical coherence tomography. In some of the foregoing embodiments, determining the functional feature estimate comprises applying at least the following equations: m=[0.054*(ec*1.32)]+0.9; b=[−1.5*(ec*1.32)]−14.8; gc={[(s−1)−b]/m}+4.7; SAPrgc=Σ10̂(gc*0.1), wherein ec comprises the eccentricity and s comprises the sensitivity from standard automated perimetry data. In some of the foregoing embodiments, determining the functional feature estimate comprises applying at least the following equations: d=(−0.007*age)+1.4; c=(−0.26*MD)+0.12; a=average RNFL thickness*10870*d; OCTrgc=10̂[(log(a)*10−c)*0.1], wherein age is the age of the patient and MD comprises a mean deviation. In some of the foregoing embodiments, determining an index comprises applying at least the following formula: wrgc=(1+MD/30)*OCTrgc+(−MD/30)*SAPrgc, wherein wrgc comprises at least a portion of the index. In some of the foregoing embodiments, the method further comprises relating the index to age and optic disc area in a population. In some of the foregoing embodiments, the method further comprises performing an optical coherence tomography analysis, such as a spectral domain optical coherence tomography analysis, on an eye. In some of the foregoing embodiments, the method further comprises performing standard automated perimetry analysis of an eye. In some of the foregoing embodiments, the method further comprises advising a subject whether or not they have glaucoma based on the value of the index. In some of the foregoing embodiments, the method further comprises advising a subject regarding progression of glaucoma based on the value of the index. In some of the foregoing embodiments, the method further comprises receiving data from a device selected from the group consisting of a wired device, a wireless device, a plug-in device, a computer and a combination of any of the foregoing devices, and external input including manual, auditory, and visual sources.

Certain embodiments contemplate a system for detecting glaucoma or assessing the progression of glaucoma, comprising: means for receiving a plurality of structural feature data; means for determining a structural feature estimate; means for receiving a plurality of functional feature data; means for determining a functional feature estimate; and means for determining an index based on a weighted combination of the structural feature estimate and the functional feature estimate. In some of the foregoing embodiments, the plurality of functional feature data comprises standard automated perimetry data. In some of the foregoing embodiments, the plurality of structural feature data comprises optical coherence tomography data, such as spectral domain optical coherence tomography data. In some of the foregoing embodiments, the plurality of structural feature data comprises estimates of the number of RGC axons from RNFL thickness measurements obtained by optical coherence tomography. In some of the foregoing embodiments, the means for determining a functional feature applies at least the following equations: m=[0.054*(ec*1.32)]+0.9; b=[−1.5*(ec*1.32)]−14.8; gc={[(s−1)−b]/m}+4.7; SAPrgc=E 10̂(gc*0.1), wherein ec comprises the eccentricity and s comprises the sensitivity from standard automated perimetry data. In some of the foregoing embodiments, the means for determining a structural feature estimate applies at least the following equations: d=(−0.007*age)+1.4; c=(−0.26*MD)+0.12; a=average RNFL thickness*10870*d; OCTrgc=10̂[(log(a)*10−c)*0.1], wherein age is the age of the patient and MD comprises a mean deviation. In some of the foregoing embodiments, the means for determining an index applies at least the following formula: wrgc=(1+MD/30)*OCTrgc+(−MD/30)*SAPrgc, wherein wrgc comprises at least a portion of the index. In some of the foregoing embodiments, the system further comprises a regression module, the regression module configured to relate the index to age and optic disc area in a population. In some of the foregoing embodiments, the system comprises a device selected from the group consisting of a wired device, a wireless device, a plug-in device, a computer and a combination of any of the foregoing devices or wherein said system receives data from a source selected from the group consisting of an external source, a manual source, an auditory source, and a visual source.

Certain embodiments contemplate a method for diagnosing glaucoma, staging glaucoma, or assessing glaucoma progression or rate of change over time comprising obtaining both structure measurements or scores and function measurements or scores to give an single index useful for said diagnosis of glaucoma, staging of glaucoma, or assessment of glaucoma progression or rate of change over time. Certain embodiments instead contemplate a method for obtaining a combined index of structure and function in an eye comprising performing both a retinal ganglion cell (RGC) count estimate from standard automated perimetry (SAP) and optical coherence tomography (OCT), such as spectral domain optical coherence tomography. In some of the foregoing embodiments, the index is determined as follows: wrgc=(1+MD/30)*OCTrgc+(−MD/30)*SAPrgc, where wrgc represents the final combined estimate of RGC counts from standard automated perimetry (SAP) and optical coherence tomography (OCT). MD represents the mean deviation obtained from standard automated perimetry and is used as a weighting variable. In some of the foregoing embodiments, wrgc represents the combined estimate of RGC count obtained from structure and function. In some of the foregoing embodiments, wrgc is used to stage and detect glaucomatous damage by comparing the values of a specific eye to those of healthy subjects. In some of the foregoing embodiments, low values of wrgc indicate glaucomatous damage. In some of the foregoing embodiments, wrgc values are evaluated over time to assess glaucomatous progression.

Certain embodiments contemplate a device that integrates data according to any one of the above-described methods to produce the index score. In some of the foregoing embodiments the device comprises one or more of the devices selected from the group consisting of a computer, a personal electronic device, a calculator, a communications device, and a dedicated integration station. In some of the foregoing embodiments the device receives data from a source selected from the group consisting of a wired source, a wireless source, a plug-in source, a computer, an external source, a manual source, an auditory source, a visual source and a combination of any of the foregoing sources.

Certain embodiments contemplate a method for determining a number of retinal ganglion cells (RGC) in an eye, comprising: administering a structural feature test to a patient to determine structural data; administering a functional feature test to a patient to determine functional data; determining a structural feature estimate based on the structural data; determining a functional feature estimate based on the functional data; determining an index based on a weighted combination of the structural feature estimate and the functional feature estimate. In some of the foregoing embodiments, the functional feature data comprises standard automated perimetry data. In some of the foregoing embodiments, the structural feature data comprises optical coherence tomography data, such as spectral domain optical coherence tomography data. In some of the foregoing embodiments, administering a structural feature test comprises estimating the number of RGC axons from RNFL thickness measurements obtained by optical coherence tomography. In some of the foregoing embodiments, determining a functional feature estimate comprises applying at least the following equations: m=[0.054*(ec*1.32)]+0.9; b=[−1.5*(ec*1.32)]−14.8; gc={[(s−1)−b]/ m}+4.7; SAPrgc=Σ10̂(gc*0.1), wherein ec comprises the eccentricity and s comprises the sensitivity from standard automated perimetry data. In some of the foregoing embodiments, determining a structural feature estimate comprises applying at least the following equations: d=(−0.007*age)+1.4; c=(−0.26*MD)+0.12; a=average RNFL thickness*10870*d; OCTrgc=10̂[(log(a)*10−c)*0.1], wherein age is the age of the patient and MD comprises a mean deviation. In some of the foregoing embodiments, determining an index comprises applying at least the following formula: wrgc=(1+MD/30)*OCTrgc+(−MD/30)*SAPrgc, wherein wrgc comprises at least a portion of the index. In some of the foregoing embodiments, the method further comprises relating the index to age and optic disc area in a population. In some of the foregoing embodiments, the method further comprises performing an optical coherence tomography analysis, such as a spectral domain optical coherence tomography analysis, on an eye. In some of the foregoing embodiments, the method further comprises performing standard automated perimetry analysis of an eye. In some of the foregoing embodiments, the method further comprises advising a subject whether or not they have glaucoma based on the value of the index. In some of the foregoing embodiments, the method further comprises advising a subject regarding progression of glaucoma based on the value of the index. In some of the foregoing embodiments, the method further comprises receiving data from a device selected from the group consisting of a wired device, a wireless device, a plug-in device, a computer and a combination of any of the foregoing devices or from a source selected from the group consisting of an external source, a manual source, an auditory source, a visual source or a combination of any of the foregoing sources. In some of the foregoing embodiments, the method, system or computer readable medium further comprises structural feature data comprising spectral domain optical coherence tomography data. In some of the foregoing embodiments, the method, system or computer readable medium further comprises administering a structural feature test comprising estimating the number of RGC axons from RNFL thickness measurements obtained by spectral domain optical coherence tomography. In some of the foregoing embodiments, the method, system or computer readable medium further comprises performing a spectral domain optical coherence tomography analysis on an eye. In some of the foregoing embodiments, the method, system or computer readable medium includes where the structural feature data comprises time domain optical coherence tomography data. In some of the foregoing embodiments, the method, system or computer readable medium further comprises administering a structural feature test comprises estimating the number of RGC axons from RNFL thickness measurements obtained by time domain optical coherence tomography.

### BRIEF DESCRIPTION OF THE DRAWINGS

FIG. 1 is a diagram of a scatterplot illustrating the relationship between the number of retinal ganglion cells (RGC) derived from standard automated perimetry (SAP) sensitivity data and the number of RGCs estimated from analysis of the retinal nerve fiber layer by optical coherence tomography (OCT).

FIG. 2 is a diagram of three histograms illustrating the distribution of the number of estimated retinal ganglion cells according to the different diagnostic categories.

FIG. 3 is a diagram illustrating a relationship between the weighted estimate of number of retinal ganglion cells (RGC) and age. A locally weighted scatterplot smoothing (lowess) shows that a linear regression fits the data well.

FIG. 4 is a diagram of boxplots illustrating the distribution of the values of the combined index of structure and function (CSFI) according to the different diagnostic categories.

FIG. 5 is a diagram of receiver operating characteristic (ROC) curves for discriminating between perimetric glaucoma and healthy eyes (left) and between preperimetric glaucoma and healthy eyes (right). ROC curves are shown for the parameters CSFI (combined index of structure and function), average retinal nerve fiber layer (RNFL) thickness and VFI (Visual Field Index).

FIG. 6 is a diagram of A. Scatterplot illustrating the relationship between mean deviation (MD) and the CSFI (combined index of structure and function) with superimposed locally weighted scatterplot smoothing (lowess). B. Scatterplot illustrating the relationship between MD and average retinal nerve fiber layer (RNFL) thickness with superimposed lowess. There is much more scatter around the lowess curve for the average thickness compared to the CSFI.

FIG. 7 is a diagram of an eye with preperimetric glaucoma included in the study. The eye had evidence of progressive optic disc change on stereophotographs (superior and inferior rim thinning), but still presented with visual fields that were statistically within normal limits. Results of the optical coherence tomography (OCT) exam show pronounced retinal nerve fiber layer thinning with average thickness of 68 μm, compatible with the changes seen on optic disc photographs. The combined index of structure and function (CSFI) was 39%, indicating a loss of 39% of retinal ganglion cells compared to the age expected number.

FIG. 8 is a diagram of two eyes with advanced glaucoma, the superior one shows mean deviation (MD) of −15.12 dB and the inferior one, MD of −23.61. Despite the important differences in visual field damage between the two cases, the optical coherence tomography results were similar in the two eyes with the same value of average retinal nerve fiber layer (RNFL) thickness of 50 μm. The combined index of structure and function (CSFI) shows markedly different results between the eyes, with values of 74% for the former and 85% for the latter.

FIG. 9 is a diagram of a scatterplot illustrating the relationship between estimates of the number of retinal ganglion cells (RGC) obtained by standard automated perimetry (SAP) and optical coherence tomography (OCT).

FIG. 10 is a diagram of a histogram of the estimates of baseline retinal ganglion cell (RGC) number combining structure and function measurements in the 213 eyes of the study group.

FIG. 11 is a diagram of a proportional Venn diagram illustrating the number of eyes detected as progressing according to the rates of retinal ganglion cell (RGC) loss, optical coherence tomography (OCT) average thickness parameter and standard automated perimetry visual field index (VFI).

FIG. 12 is a diagram of an eye detected as having progression during follow-up according to the rate of retinal ganglion cell (RGC) loss with a slope of −51761 cells/year (P<0.05). The eye also had progression according to the Visual Field Index (VFI) with slope of −2.0%/year and the optical coherence tomography parameter average thickness (slope of −2.8 μm/year).

FIG. 13 is a diagram of an eye detected as progressing by the rate of retinal ganglion cell loss with a slope of −45567 cells/year (P<0.05), but not by the Visual Field Index. The eye had early glaucomatous damage and showed progressive neuroretinal rim thinning as seen on the optic disc stereophotographs. The optical coherence tomography parameter average thickness showed a statistically significant slope of −3.2 μm/year.

FIG. 14 is a diagram of an eye detected as progressing by the rate of retinal ganglion cell (RGC) loss with a slope of −15397 cells/year (P<0.05), but not by the optical coherence tomography average thickness parameter. The eye had advanced visual field loss and a statistically significant slope of change with the Visual Field Index (−2.3%/year).

FIG. 15 is a diagram of boxplots illustrating the distribution of estimated retinal ganglion cell (RGC) counts in glaucomatous eyes with early visual field defects and control healthy eyes.

FIG. 16 is a diagram illustrating the distribution of estimated percent losses of retinal ganglion cells (RGCs) in the glaucomatous eyes with early visual field defects.

FIG. 17 is a diagram illustrating the receiver operating characteristic curves for discriminating glaucomatous eyes with early visual field defects from healthy eyes for the estimated retinal ganglion cell (RGC) counts and the average retinal nerve fiber layer (RNFL) thickness parameter.

FIG. 18 is a diagram illustrating grayscale and pattern deviation plots for the visual fields for one of the glaucomatous eyes in the study. The normal baseline visual field is shown along with the 3 consecutive abnormal visual fields during follow-up. The remaining normal visual fields between baseline and the first abnormal field were omitted. Estimates of retinal ganglion cell (RGC) counts were calculated using data from the first abnormal visual field (Jun. 10, 2011) and the spectral domain optical coherence tomography (Jul. 9, 2011). The eye had an estimated RGC count of 520 950 cells at the time of development of the initial visual field defect on standard automated perimetry, corresponding to a 43% RGC loss compared with the healthy group. This is in agreement with extensive neuroretinal rim loss seen on the optic disc photograph. MD=mean deviation; PSD=pattern standard deviation; RNFL=retinal nerve fiber layer.

FIG. 19 is a diagram illustrating grayscale and pattern deviation plots for the visual fields for one of the glaucomatous eyes in the study. The normal baseline visual field is shown along with the 3 consecutive abnormal visual fields during follow-up. The remaining normal visual fields between baseline and the first abnormal field were omitted. Estimates of retinal ganglion cell (RGC) counts were calculated using data from the first abnormal visual field (May 13, 2010) and the spectral domain optical coherence tomography (SD-OCT) (Jul. 20, 2010). The eye had an estimated RGC count of 800 369 at the time of development of the initial visual field defect, which corresponded to a 12% RGC loss compared with the healthy group. The optic disc photograph shows inferior neuroretinal rim thinning in agreement with inferior retinal nerve fiber layer (RNFL) loss detected by SD-OCT. MD=mean deviation; PSD=pattern standard deviation.

FIG. 20 is a diagram illustrating an example of an eye with preperimetric glaucomatous damage. The eye had evidence of progressive optic disc damage on stereophotographs (superior and inferior rim thinning), but still had a visual field exam with parameters within statistically normal limits. Results of the spectral-domain optical coherence tomography (SDOCT) exam show superior and inferior retinal nerve fiber layer (RNFL) thinning with a global RNFL thickness of 62 μm. The combined structure and function index (CSFI) was 41%, indicating a loss of 41% of retinal ganglion cells compared to the age-expected normal number. RNFL: retinal nerve fiber layer; CSFI: combined structure and function index; VFI: visual field index; MD: mean deviation; PSD: pattern standard deviation; GHT: glaucoma hemifield test; dB: decibels.

FIG. 21 is a diagram illustrating an example of two eyes with advanced glaucoma (a and b). Both eyes had identical measurements of RNFL thickness of 56 μm, despite widely different degrees of visual field loss. One eye had a MD of −13.33 dB (a) and the other one had a MD of −24.47 dB (b). The CSFI showed clearly different results for the two eyes, with values of 74% for a and 91% for b. RNFL: retinal nerve fiber layer; MD: mean deviation; CSFI: combined structure and function index. VFI: visual field index; PSD: pattern standard deviation; GHT: glaucoma hemifield test; dB: decibels.

FIG. 22 is a diagram illustrating an example of an eye detected as progressing by the rate of retinal ganglion cell loss with a slope of −52 902 cells/year (P<0.05), and by global retinal nerve fiber layer (RNFL) thickness with a slope of −3.2 μm/year. Assessment of rates of visual field change with the visual field index was unable to detect significant change (P>0.05). RNFL: retinal nerve fiber layer. The eye had clear progression confirmed by longitudinal assessment of optic disc stereophotographs.

FIG. 23 is a diagram illustration an example of an eye detected as progressing by the rate of retinal ganglion cell loss with a slope of −65 990 cells/year (P<0.05); and by the rate of visual field loss, with a slope of −1.8%/year (P<0.05). The optical coherence tomography parameter global retinal nerve fiber layer (RNFL) thickness did not show a statistically significant slope (P>0.05).

FIG. 24 is a block diagram of one embodiment of a configuration for operating a system and method to diagnose glaucoma, stage glaucoma, or assess a glaucoma progression or rate of change over time.

FIG. 25 is a diagram of one embodiment of a configuration of modules to determine an index estimating a number of retinal ganglion cells in an eye using a system such as that shown in FIG. 24.

FIG. 26 is a flow diagram of one embodiment to detect glaucoma or assess the progression of glaucoma using a system such as that shown in FIG. 24.

### DETAILED DESCRIPTION OF THE PREFERRED EMBODIMENTS

A new index to estimate glaucoma severity based on a combination of functional measurements and structural measurements is described herein. In some embodiments, the functional measurements may be obtained by techniques such as standard automated perimetry (SAP). In some embodiments, the structural measurements may be obtained by techniques such as optical coherence tomography. For example, in some embodiments the structural measurements may be obtained by spectral domain optical coherence tomography. It is shown that the index performs well in discriminating diseased from non-diseased patients and provides a better estimate of the stage of glaucoma severity compared to the isolated use of functional or structural measures.

In some embodiments, a system and method can be used to determine an index estimating a number of retinal ganglion cells (RGC) in an eye, including administering a structural feature test to a patient to determine structural data; administering a functional feature test to a patient to determine functional data; determining a structural feature estimate based on the structural data; determining a functional feature estimate based on the functional data; and determining the index based on a weighted combination of the structural feature estimate and the functional feature estimate. In some embodiments, the functional feature data includes standard automated perimetry data, and the structural feature data includes optical coherence tomography data, such as spectral domain optical coherence tomography data. Administering a structural feature test can include estimating the number of RGC axons from RNFL thickness measurements obtained by optical coherence tomography, such as spectral domain optical coherence tomography.

In some embodiments, determining a functional feature estimate can include applying at least the following equations: m=[0.054*(ec*1.32)]+0.9; b=[−1.5*(ec*1.32)]−14.8; gc={[(s−1)−b]/m}+4.7; SAPrgc=Σ10̂(gc*0.1), where ec comprises the eccentricity and s comprises the sensitivity from standard automated perimetry data. Determining a structural feature estimate can include applying at least the following equations: d=(−0.007*age)+1.4; c=(−0.26*MD)+0.12; a=average RNFL thickness*10870*d; OCTrgc=10̂[(log(a)*10−c)*0.1], where age is the age of the patient and MD comprises a mean deviation. Determining an index can include applying at least the following formula: wrgc=(1+MD/30)*OCTrgc+(−MD/30)*SAPrgc, where wrgc comprises at least a portion of the index. The system and method can further include relating the index to age and optic disc area in a population, and can further include performing an optical coherence tomography analysis, such as a spectral domain optical coherence tomography analysis, on an eye. The system and method can further include performing standard automated perimetry analysis of an eye, and can further include advising a subject whether or not they have glaucoma based on the value of the index. The system and method can further include advising a subject regarding progression of glaucoma based on the value of the index.

A first observational study was conducted as follows. Participants from this study were included in two prospective longitudinal studies designed to evaluate optic nerve structure and visual function in glaucoma (the African Descent and Glaucoma Evaluation Study [ADAGES] and the Diagnostic Innovations in Glaucoma Study [DIGS]). The 3-site ADAGES collaboration includes the Hamilton Glaucoma Center at the Department of Ophthalmology, University of California-San Diego (UCSD) (data coordinating center), the New York Eye and Ear Infirmary and the Department of Ophthalmology, University of Alabama, Birmingham (UAB). Although the DIGS includes only patients recruited at UCSD, the protocols of the two studies are identical. The institutional review boards at all 3 sites approved the study methodology, which adhered to the tenets of the Declaration of Helsinki and to the Health Insurance Portability and Accountability Act. Methodological details have been described previously.¹⁹

At each visit during follow-up, subjects underwent a comprehensive ophthalmologic examination including review of medical history, best-corrected visual acuity, slit-lamp biomicroscopy, intraocular pressure (IOP) measurement, gonioscopy, dilated fundoscopic examination, stereoscopic optic disc photography, and automated perimetry using Swedish Interactive Threshold Algorithm (SITA Standard 24-2). Only subjects with open angles on gonioscopy were included. Subjects were excluded if they presented with a best-corrected visual acuity less than 20/40, spherical refraction outside ±5.0 diopters and/or cylinder correction outside 3.0 diopters, or any other ocular or systemic disease that could affect the optic nerve or the visual field.

The study included 333 eyes of 246 glaucoma patients diagnosed based on evidence of presence of repeatable glaucomatous visual field defects or documented history of progressive glaucomatous optic neuropathy. From the 333 eyes, 295 had evidence of glaucomatous visual field defects based on repeatable abnormal visual field test results defined as a pattern standard deviation (PSD) outside of the 95% normal confidence limits, or a Glaucoma Hemifield Test result outside normal limits. An additional group of 38 eyes had evidence of progressive glaucomatous change in the appearance of the optic disc as assessed by masked grading of simultaneous stereoscopic optic disc photographs (TRC-SS; Topcon Instrument Corp of America, Paramus, N.J., USA), despite absence of statistically significant visual field losses. The evidence of progressive glaucomatous damage had to be present before the imaging test date and the details of the methodology employed to grade optic disc photographs at the UCSD Optic Disc Reading Center have been provided elsewhere.^{20, 21} This latter group was used to assess the ability of the proposed staging system to quantify damage in patients with confirmed preperimetric glaucoma.

The control group consisted of 330 eyes from 171 healthy participants. These subjects were recruited from the general population and were required to have a normal ophthalmologic examination and IOP below 22 mmHg in both eyes, but results of visual field tests were not used as inclusion or exclusion criteria.

### Visual Field Testing

All patients underwent SAP testing using SITA-standard 24-2 strategy less than 6 months apart from imaging. All visual fields were evaluated by the UCSD Visual Field Assessment Center (VisFACT).²² Visual fields with more than 33% fixation losses or false-negative errors, or more than 15% false-positive errors were excluded. The only exception was the inclusion of visual fields with false-negative errors of more than 33% when the field showed advanced disease (MD lower than −12 dB). Visual fields exhibiting a learning effect (e.g., initial tests showing consistent improvement on visual field indexes) were also excluded. Visual fields were further reviewed for the following artifacts: lid and rim artifacts, fatigue effects, inappropriate fixation, evidence that the visual field results were due to a disease other than glaucoma (such as homonymous hemianopia), and inattention. The VisFACT requested repeats of unreliable visual field test results, and these were obtained whenever possible.

### Spectral-Domain OCT

The Cirrus HDOCT (software version 5.2, Carl Zeiss Meditec Inc., Dublin) was used to acquire RNFL measurements in the study. It uses a superluminescent diode scan with a center wavelength of 840 nm and an acquisition rate of 27 000 A-scans per second at an axial resolution of 5 μm. The protocol used for RNFL thickness evaluation was the optic disc cube. This protocol is based on a 3-dimensional scan of a 6×6 mm² area centered on the optic disc where information from a 1024 (depth)×200×200-point parallelepiped is collected. Then, a 3.46-mm diameter circular scan (10.87 mm length) is automatically placed around the optic disc, and the information about parapapillary RNFL thickness is obtained. Because information from the whole region is obtained, it is possible to modify the position of the scan after the exam is taken. To be included, all images were reviewed for non-centered scans and had to have signal strength >6, the absence of movement artifacts, and good centering around the optic disc.

### Combined Structure and Function Index

The development of the combined index of structure and function to measure disease severity was based on previous work by Harwerth and colleagues² on the development and validation of a model linking structure and function in glaucoma. Based on experimental studies in monkeys, the authors first derived an empirical model relating sensitivity measurements in SAP to histological RGC counts as a function of retinal eccentricities. The experimental results were then translated to clinical perimetry in humans. The following formulas were proposed to estimate the number of RGC somas in an area of the retina corresponding to a specific SAP test field location at eccentricity ec with sensitivity s in dB:

m=[0.054*(ec*1.32)]+0.9

b=[−1.5*(ec*1.32)]−14.8

gc={[(s−1)−b]/m}+4.7

SAPrgc=Σ10̂(gc*0.1)

Where m and b represent the slope and intercept, respectively, of the linear function relating ganglion cell quantity (gc) in decibels to the visual field sensitivity (s) in decibels at a given eccentricity (ec). By applying the above formulas, one can obtain a SAP-derived estimate of the total number of RGCs (SAPrgc) by adding the estimates from all locations in the visual field. The structural part of the model consisted in estimating the number of RGC axons from RNFL thickness measurements obtained by optical coherence tomography, such as spectral domain optical coherence tomography. The model took into account the effect of aging in the axonal density and the effect of disease severity on the relationship between the neuronal and non-neuronal components of the RNFL thickness estimates obtained by OCT. To derive the total number of RGC axons from the global RNFL thickness measurement obtained by OCT (OCTrgc), one can apply the following formulas:

d=(−0.007*age)+1.4

c=(−0.26*MD)+0.12

a=average RNFL thickness*10870*d

OCTrgc=10̂[(log(a)*10−c)*0.1]

Where d corresponds to the axonal density (axons per micrometers squared) and c is a correction factor for the severity of disease to consider remodeling of the RNFL axonal and nonaxonal composition. The above calculations allow one to estimate the number of RGCs from two sources, one functional and one structural, and a strong relationship was demonstrated between the two estimates in external validation cohorts. However, although Harwerth et al proposed a model linking structure and function, no attempt was made to develop an index combining structural and functional estimates that could be clinically used to stage glaucoma severity. The following calculations may be used to develop such an index. In order to derive a combined index, the estimates of RGC numbers obtained from SAP and OCT were simply averaged, but weighting according to severity of disease. As clinical perimetry and imaging tests accuracies have been proposed to be inversely related to disease severity, we propose a weighted scale combining the estimates of RGC numbers from both tests:

wrgc=(1+MD/30)*OCTrgc+(−MD/30)*SAPrgc

The weights were chosen to reflect the inverse relationship with disease severity of SAP and OCT estimates, along the scale of MD values ranging from 0 to −30dB. After estimates of wrgc were obtained, a linear regression model was run to relate wrgc estimates to age and optic disc area in the normal control population. The purpose was to develop a model to predict expected RGC numbers according to age and optic disc area. In order to avoid model overfitting, the regression parameters were obtained using only half of the normal eyes (development sample). After the expected number of RGCs was calculated for each eye, an estimate of the percent RGC loss for each eye was obtained by subtracting measured from estimated RGC numbers. The percent estimate of RGC loss should reflect an estimate of glaucomatous damage obtained by combining data from structural and functional measurements (CSFI, combined structure-function index), as calculated below:

CSFI=[(expected RGC number−wrgc)/(expected RGC number)]*100

### Statistical Analysis

The performance of the CSFI for discriminating glaucoma from normal eyes and the different stages of disease was compared to those of other indexes previously used to stage disease severity such as MD and the Visual Field Index (VFI), as well as to the SDOCT parameter average RNFL thickness. Receiver operating characteristic (ROC) curves were built, and the area under the ROC curves (AUC) was used to summarize the diagnostic accuracy for each parameter. Perimetric and preperimetric glaucomatous eyes were compared to normal eyes in the validation sample, e.g., excluding the eyes previously used to obtain the regression parameters described above. An AUC equal to 1 represents perfect discrimination, whereas an AUC of 0.5 represents chance discrimination. AUCs and 95% confidence intervals were obtained for each parameter after adjusting for age. A bootstrap resampling procedure (n=1000 resamples) was used to derive confidence intervals. Age adjustment was performed using a ROC regression model, as previously described. The model is able to adjust for the differences in variables between control and cases by fitting a linear regression of the marker distribution on the adjustment variables among controls. Standardized residuals based on this fitted linear model are used in place of the marker values for cases and controls. To account for the potential correlation between eyes, the cluster of data for the study subject was considered as the unit of resampling when calculating standard errors. This procedure has been previously used to adjust for the presence of multiple correlated measurements from the same unit.²³

All statistical analyses were performed with commercially available software (Stata version 12; StataCorp, College Station, Tex.). The alpha level (type I error) was set at 0.05.

### Results

From the 333 glaucomatous eyes, 295 (89%) had perimetric glaucoma and 38 (11%) had preperimetric glaucoma. The eyes were compared to 165 eyes from 85 healthy subjects included in the validation sample. The mean ages of perimetric glaucoma and preperimetric glaucoma participants were 69±11 and 66±10, respectively. They were both significantly higher than that of control subjects (60±11; P<0.01 for both comparisons). Age differences were adjusted for in the ROC analyses.

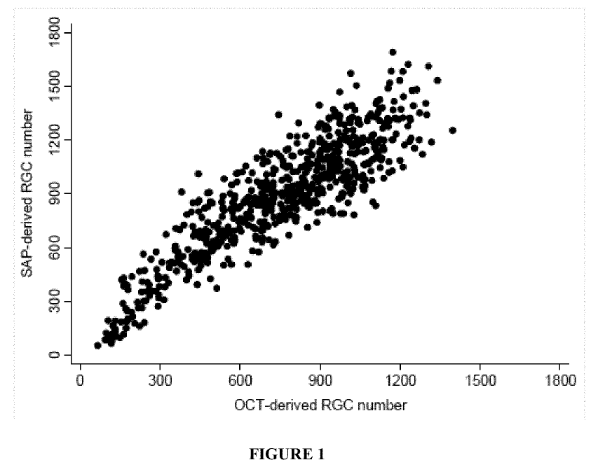

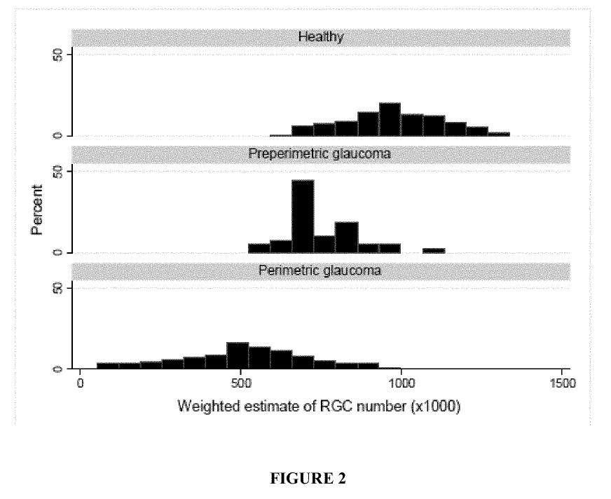

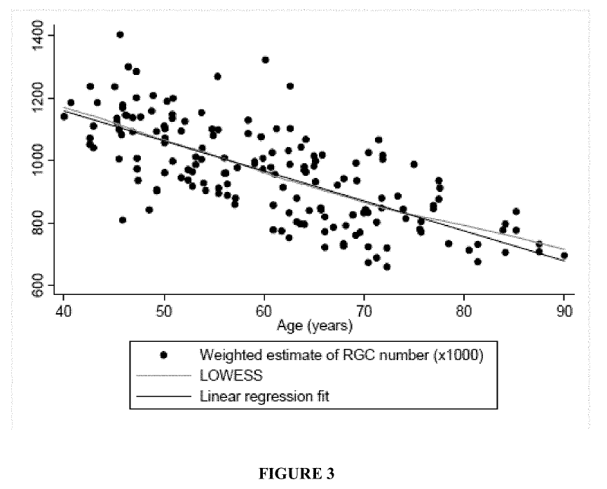

Table 1 shows estimates of the different parameters obtained in the study. There was a strong correlation between RGC estimates obtained from SAP and OCT data in the eyes included in the study (r=0.89; P<0.001) (FIG. 1). FIG. 2 shows histograms of calculated weighted estimates of RGC numbers combining structural and functional tests (wrgc), according to the diagnostic categories. The mean estimated number of RGCs in the group with perimetric glaucoma was 524,545 compared to 748,731 in the preperimetric group and 973,120 in normal eyes. The results of the linear regression model relating estimated RGC numbers to age and optic disc area in the normal eyes from the development sample are presented on Table 2. There was a significant relationship between RGC number and age, with an estimated loss of 9,249 RGCs per year older in normal subjects (FIG. 3). Also, each 0.1 mm² larger optic disc area corresponded to an increase in 11,607 RGCs.

TABLE 1
Mean values of the different parameters calculated in the study in perimetric
glaucoma, preperimetric glaucoma and healthy eyes.*

| | | | | P | P |
|---|---|---|---|---|---|
| | Perimetric | Preperimetric | | (Perimetric | (Preperimetric |
| | glaucoma | glaucoma | Healthy | glaucoma vs. | glaucoma vs. |
| | (n = 295) | (n = 38) | (n = 165) | healthy) | healthy |
| MD^{¶}, dB | −4.01 (−1.79, −9.40) | −0.32 (−1.33, 0.47) | 0.17 (−0.85, 1.05) | <0.001 | 0.015 |
| PSD^{¶}, dB | 4.80 (2.59, 9.77) | 1.52 (1.41, 1.76) | 1.60 (1.34, 1.85) | <0.001 | 0.407 |
| VFI^{¶}, % | 92 (77, 97) | 99 (99, 100) | 99 (99, 100) | <0.001 | 0.825 |
| Average thickness, | 69 (13) | 78 (10) | 94 (9) | <0.001 | <0.001 |
| μm | | | | | |
| SAPrgc, ×1000 cells | 660 (277) | 944 (148) | 1075 (208) | <0.001 | <0.001 |
| OCTrgc, ×1000 cells | 502 (221) | 749 (107) | 977 (156) | <0.001 | <0.001 |
| wgc, ×1000 cells) | 525 (210) | 749 (105) | 973 (154) | <0.001 | <0.001 |
| CSFI, % | 41 (22) | 17 (10) | 4 (7) | <0.001 | <0.001 |

*Values are given as mean (standard deviation), unless otherwise indicated
^{¶}Median (first quartile, third quartile)
MD—mean deviation;
PSD—pattern standard deviation;
VFI—visual field index;
SAPrgc—Number of retinal ganglion cells estimated from SAP sensitivity values;
OCTrgc—number of retinal ganglion cells estimated from optical coherence tomography data;
wgc—weighted estimated of the number of retinal ganglion cells;
CSFI—combined index of structure and function.

TABLE 2
Results of the linear regression model evaluating
the association between the weighted number of retinal
ganglion cells and age and optic disc area in healthy eyes.*

| Parameter | Coefficient | 95% CI | P |
|---|---|---|---|
| Age, per year older | −9249 | −10613 to −7885) | <0.001 |
| Optic Disc Area, per 0.1 mm² | 11607 | 6077 to 17138 | <0.001 |
| larger | | | |
| Constant | 1301098 | 1163399 to 1438796 | <0.001 |

*Data from the 165 healthy eyes included in the development sample.

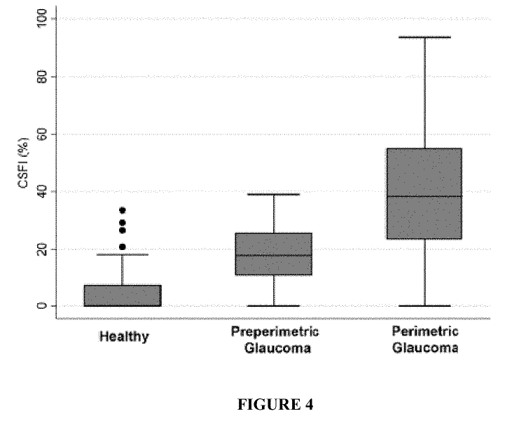

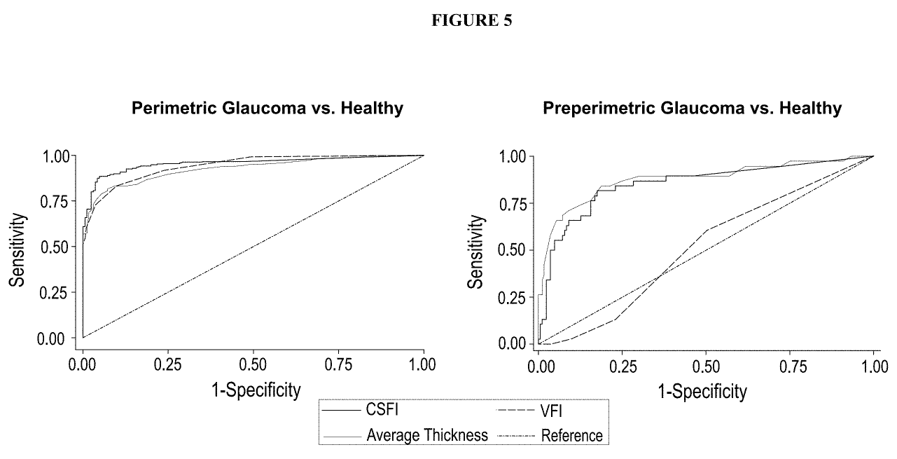

The mean CSFI, representing the mean estimated percent loss of RGCs, was 41% and 17% in the perimetric and pre-perimetric groups, respectively (P<0.001). They were also both significantly higher than the mean CSFI in the normal group (P<0.001) (Table 1). FIG. 4 shows a boxplot graph of the CSFI values according to diagnostic category. Table 3 shows the areas under the ROC curves for the parameters investigated in the study. The CSFI had an ROC curve area of 0.94 to discriminate glaucomatous from normal eyes. The performance of the CSFI was superior to that of SDOCT parameter average RNFL thickness (AUC=0.92; P=0.008) and the global visual field indexes MD (AUC=0.88; P<0.001) and VFI (AUC=0.89; P<0.001). Analyses were also performed by subgroups of perimetric and preperimetric glaucoma. For detection of perimetric glaucoma, the CSFI also performed significantly better than average RNFL thickness and MD (P<0.001 for both comparisons), but not significantly different from the VFI (P=0.16) (Table 4). For detecting preperimetric glaucoma, the CSFI had an ROC curve area of 0.85, which was superior to that of the VFI (AUC=0.51; P<0.001) and MD (AUC=0.63; P<0.001). The ability to detect preperimetric glaucoma with the CSFI was similar to that of the SDOCT parameter average RNFL thickness (AUC=0.88; P=0.32). FIG. 5 shows ROC curves for the different parameters for detection of perimetric and preperimetric glaucoma.

TABLE 3
Areas under the receiver operating characteristic (ROC) curves and
standard errors for the parameters evaluated in the study.

| | | Perimetric | Preperimetric |
|---|---|---|---|
| | Glaucoma vs. | glaucoma vs. | glaucoma |
| | Healthy | Healthy | vs. Healthy |
| MD | 0.88 (0.01) | 0.92 (0.01) | 0.63 (0.05) |
| PSD | 0.88 (0.01) | 0.94 (0.01) | 0.46 (0.05) |
| VFI | 0.89 (0.01) | 0.94 (0.01) | 0.51 (0.04) |
| Average thickness | 0.92 (0.01) | 0.93 (0.01) | 0.88 (0.04) |
| SAPrgc | 0.86 (0.02) | 0.89 (0.01) | 0.69 (0.04) |
| OCTrgc | 0.95 (0.01) | 0.96 (0.01) | 0.88 (0.03) |
| Wgc | 0.95 (0.01) | 0.96 (0.01) | 0.88 (0.03) |
| CSFI | 0.94 (0.01) | 0.96 (0.01) | 0.85 (0.04) |

MD—mean deviation;
PSD—pattern standard deviation;
VFI—visual field index;
SAPrgc—Number of retinal ganglion cells estimated from SAP sensitivity values;
OCTrgc—number of retinal ganglion cells estimated from optical coherence tomography data;
wgc—weighted estimated of the number of retinal ganglion cells;
CSFI—combined index of structure and function.

TABLE 4
Values of the parameters obtained in the study for the different stages of glaucoma
severity based on the Hodapp-Anderson-Parrish classification.

| | Early glaucoma | Moderate glaucoma | Advanced glaucoma |
|---|---|---|---|
| | (n = 189) | (n = 49) | (n = 57) |
| MD^{¶}, dB | −2.3 (−3.7, −1.0) | −8.2 (−9.7, −7.0) | −17.4 (−23.3, −14.7) |
| PSD^{¶}, dB | 3.0 (2.1, 4.6) | 9.9 (7.2, 11.6) | 11.6 (9.4, 13.6) |
| VFI^{¶}, % | 96 (93, 98) | 80 (75, 84) | 51 (32, 58) |
| Average thickness, μm | 74 (12) | 65 (10) | 57 (9) |
| SAPrgc, ×1000 cells | 812 (180) | 540 (157) | 260 (138) |
| OCTrgc, ×1000 cells | 628 (156) | 376 (82) | 193 (70) |
| wgc, ×1000 cells) | 641 (147) | 422 (82) | 227 (100) |
| CSFI, % | 28 (13) | 52 (8) | 75 (11) |

MD—mean deviation;
PSD—pattern standard deviation;
VFI—visual field index;
SAPrgc—Number of retinal ganglion cells estimated from SAP sensitivity values;
OCTrgc—number of retinal ganglion cells estimated from optical coherence tomography data;
wgc—weighted estimated of the number of retinal ganglion cells;
CSFI—combined index of structure and function.

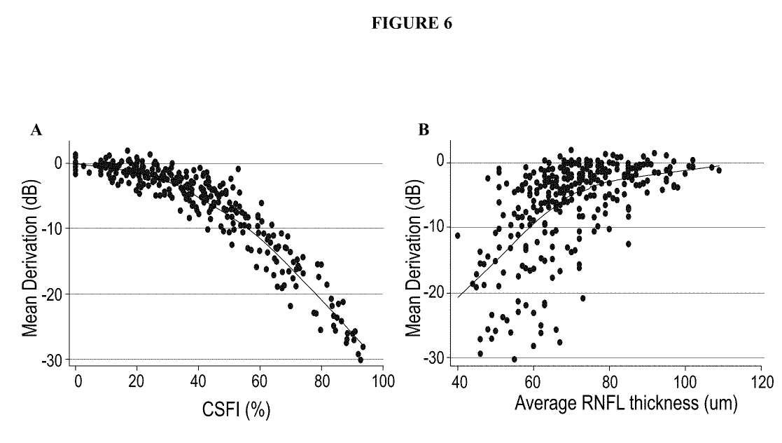

The ability of the CSFI in discriminating eyes with different stages of glaucomatous visual field loss as determined by the Hodapp-Anderson-Parrish (HAP) classification system was also evaluated. According to the HAP, from the 295 eyes with glaucomatous visual field loss, 189 had early damage, 49 had moderate and 57 had advanced. Table 4 shows the values of the parameters calculated in the study for these different severity groups. The AUC for the CSFI for separating early from moderate visual field loss was 0.94 (±0.02), compared to only 0.77 (±0.02) for the SDOCT average RNFL thickness (P<0.001). For separating moderate from advanced glaucomatous field loss, the AUC of the CSFI was 0.96 (±0.02), which was again significantly better than that for average RNFL thickness (AUC=0.70±0.05; P<0.001). The CSFI also performed better than average RNFL thickness to discriminate eyes with preperimetric glaucoma from those with early visual field loss (0.73±0.04 vs. 0.60±0.04, respectively; P<0.001). FIG. 6(a) shows the relationship between MD and CSFI whereas FIG. 6(b) shows the relationship between MD and average thickness. It can be seen that the CSFI agrees more closely with MD than the parameter average RNFL thickness in moderate and advanced stages of the disease.

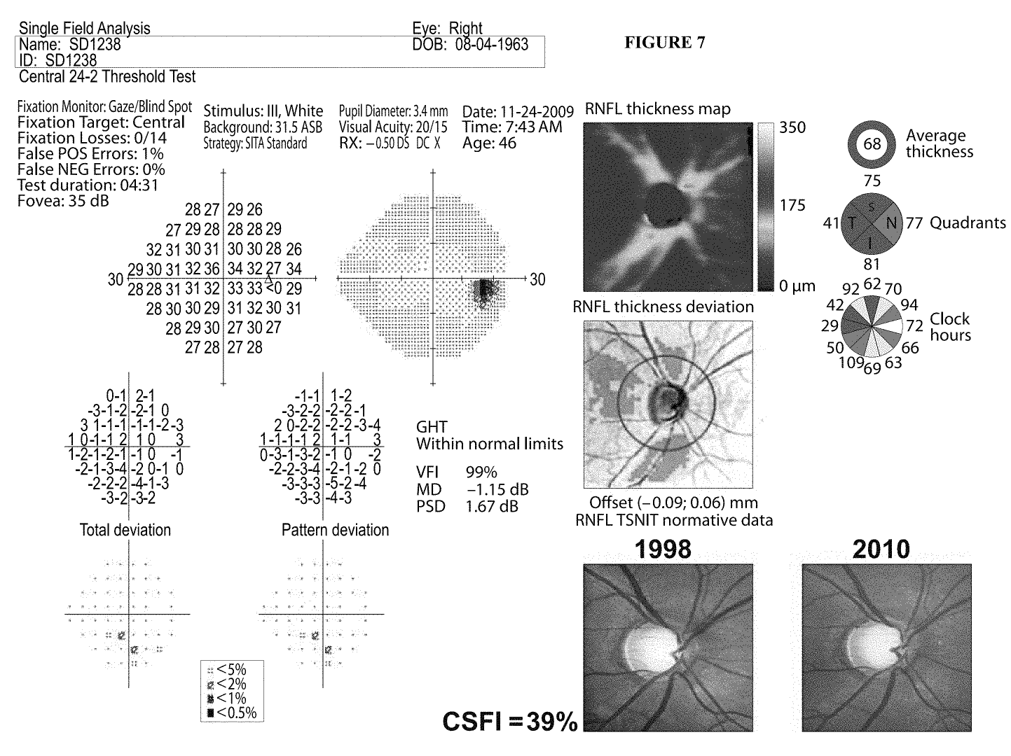

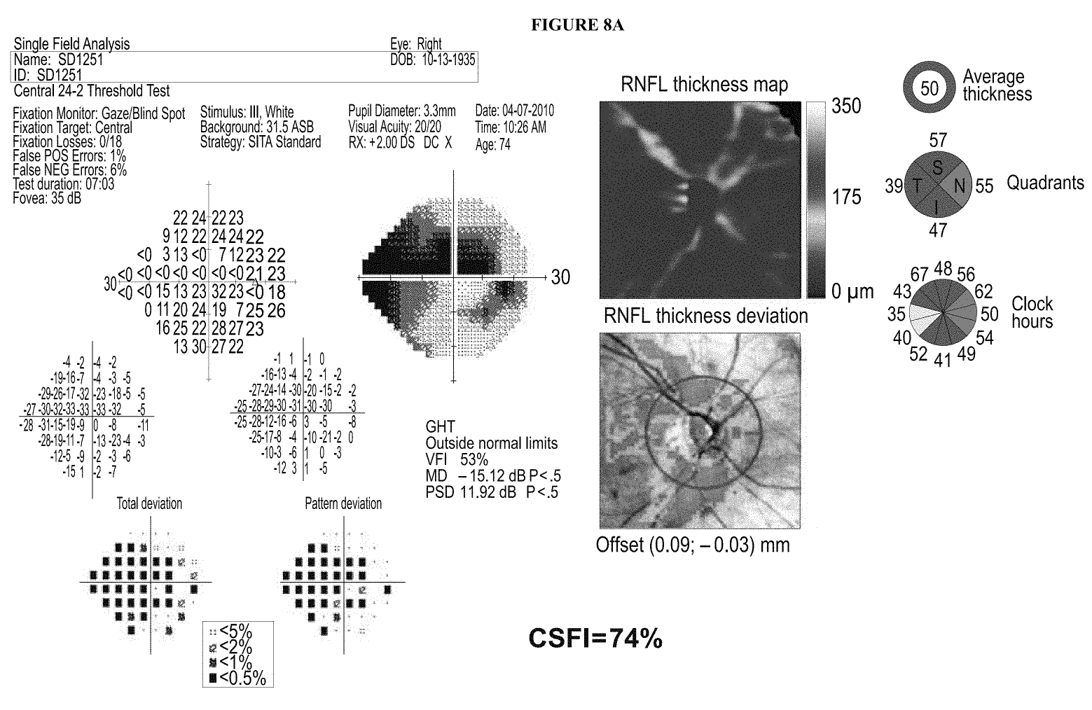

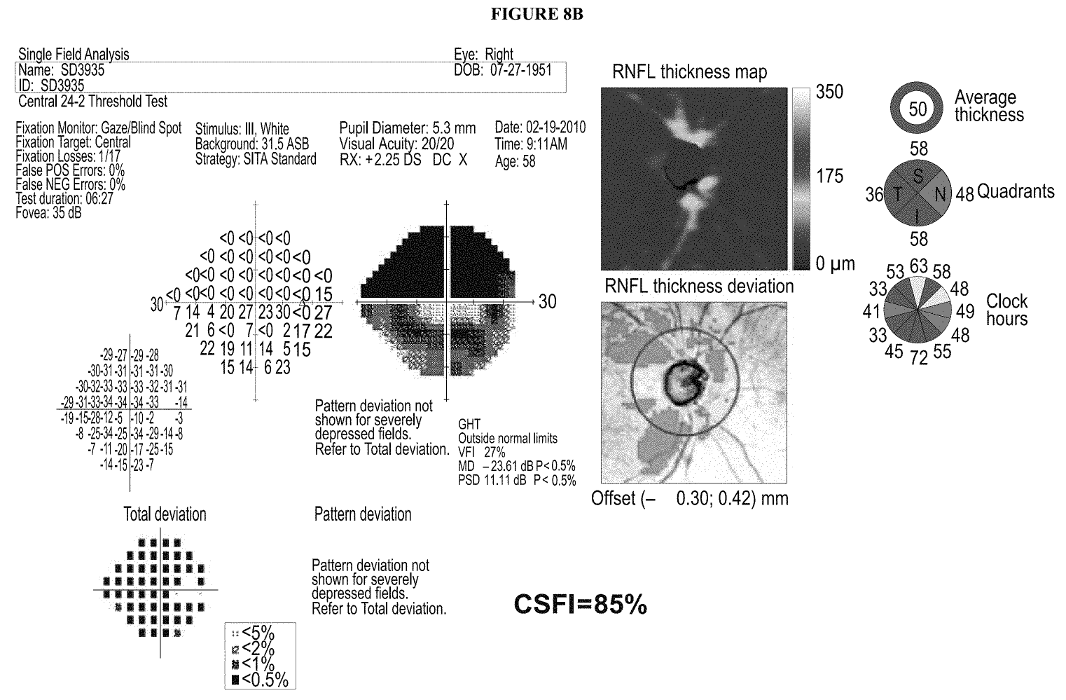

FIG. 7 illustrates a case of preperimetric glaucoma included in the study. The eye had clear evidence of documented progressive optic disc change on stereophotographs before the imaging test date but still presented with visual fields that were statistically within normal limits. Results of the SDOCT exam show pronounced RNFL thinning, with average thickness of 68 μm. The CSFI for the eye was 39%, indicating a loss of 39% of the estimated number of RGCs compared to the age expected number. FIG. 8 shows two eyes with advanced glaucoma, one with MD of −15.12dB and another with MD of −23.61. Despite the important differences in visual field damage between the two cases, SDOCT results were similar in the two eyes with the same value of average thickness of 50 μm. The CSFI clearly distinguished between the eyes with values of 74% for the former and 85% for the latter.

### Discussion

In the above study, a new index combining information on structural and functional damage in glaucoma is proposed which can be used to stage and provide diagnostic information on the disease. The index performed significantly better than isolated measures of structure and function for diagnosing pre-perimetric and perimetric glaucoma. In addition, the index also performed better in discriminating different stages of the disease, suggesting that it might also be helpful for staging and monitoring patients over time.

Several staging systems for glaucoma have been proposed in the literature.²⁴⁻²⁹ Most of them have been based solely on information extracted from visual fields. Visual field-based staging systems assume that all patients with statistically normal fields should be grouped at a single stage and, therefore, they do not differentiate whether the patient is actually a healthy subject, has suspicious findings for the disease or evidence of glaucomatous neuropathy despite absence of detectable field losses. Experimental and clinical research, however, has shown that a substantial number of RGCs may need to be lost before detectable changes are observed in the visual field.² Evidence of structural damage to the optic disc and RNFL has been demonstrated in patients with statistically normal visual fields using different imaging technologies and conventional stereophotographs.^{5, 8, 20, 21} More importantly, these structural changes have been shown to carry prognostic information, being strongly associated with risk of development of future functional losses in the disease.⁵ In our study, patients with preperimetric glaucoma had an estimated mean number of RGCs of 748,731 which was approximately 23% lower than the mean number of 973,720 cells measured in the healthy eyes included in the validation sample. Differences in the number of cells could be partially explained by age differences in the two groups. Therefore, we calculated the CSFI which corresponds to a percent estimate of loss compared to the age-expected number of RGCs. Patients with pre-perimetric glaucoma had a mean CSFI of 17% which was still significantly higher than that of healthy subjects. The diagnosis of preperimetric glaucoma in our study was based on documented evidence of progressive optic disc change in stereophotographs. Due to the wide variability of the optic nerve appearance, a single optic disc examination is frequently not diagnostic in the early stages of glaucoma.^{5, 21} In the absence of visual field loss, a diagnosis of certainty of glaucoma can only be given by demonstrating a previous history of progressive glaucomatous changes to the optic nerve. We demonstrated that the CSFI performed well in differentiating eyes with preperimetric glaucoma from healthy subjects, with an ROC curve area of 0.85, similar to what can be obtained from analysis using SDOCT average thickness.

Staging systems based on optic disc appearance or quantitative assessment of the optic disc and RNFL have also been proposed.^{27, 30} These classification systems are limited by the decreasing performance of imaging instruments to discriminate among the different stages of disease with increasing severity of damage. Sihota et al³¹ reported an area under the ROC curve of only 0.705 for discriminating early to moderate visual field losses with the OCT parameter average thickness. A weak performance was also reported in separating moderate from advanced cases with an ROC curve area of only 0.737. These values are very similar to those found in our study for the SDOCT parameter average thickness, with corresponding areas under the ROC curve of 0.77 and 0.70, respectively. Longitudinal studies have also shown an inverse relationship between disease severity and ability to detect change with imaging devices.^{15, 17, 32} These findings collectively suggest that the use of a structure-only staging system is likely to be inadequate once the patient has been diagnosed with visual field loss. In contrast, the use of a combined index of structure and function allowed excellent separation between the different stages of the disease. The CSFI had areas under the ROC curve of 0.94 to separate early from moderate loss and 0.96 for discriminating moderate from advanced loss. Although these results may seem obvious as the CSFI actually incorporates visual function information used to define severity or classifying the groups, they need to be seen in the context of the overall performance of the CSFI. The CSFI performed well not only to differentiate the different stages of glaucomatous visual field loss but also in detecting preperimetric glaucoma. Therefore, using a single index combining structure and function, we were able to detect the earliest stages of damage while retaining the ability to differentiate among the different stages of the disease in more advanced cases, a task that was poorly performed when visual field data or OCT data were used in isolation.

It is important to note that some overlap in CSFI values was seen among the different studied groups as shown on FIG. 4. However, this is a limitation inherent to any parameter assessing biologic variables and could also be related to the variability of the tests used to obtain estimates of RGC numbers. Both SAP and OCT have test-retest variability and this will obviously translate into CSFI variability. This should not have affected the comparisons performed in our study, however, it indicates the need for clinicians to obtain multiple tests to improve reproducibility, as currently performed in clinical practice.

The estimates of SAP and OCT-derived RGC numbers were based on previously published work by Harwerth and colleagues.² Using normal monkeys and monkeys with laser-induced experimental glaucoma, they showed that SAP sensitivity values can provide good estimates of the amount of histologically-measured RGC counts in the retina. These estimates agreed closely with those obtained from OCT RNFL thickness data. They showed a strong linear relationship between the number of RGC somas and axons obtained from functional and structural measures, respectively, when retinal eccentricity and appropriate measurement scales for neural and sensitivity losses were used. The linear relationship suggests that the lack of sensitivity of SAP for detection of early glaucomatous damage is most likely not the result of true structural changes occurring in the absence of functional losses, but is rather related to the logarithmic scale used for SAP sensitivity measurements, as well as the magnitude of change required to reach statistically significant levels of abnormality.^{6, 33} The logarithmic scale compresses the range of losses in early stages of the disease while expanding the range in later stages. These findings could suggest that a simple linearization of SAP data could improve detection of early damage. However, this is usually not the case. In fact, the ROC curve for detecting preperimetric glaucoma using estimates of RGC number from SAP (SAPrgc) in our study was still only 0.69, much inferior to that of RGC estimates from OCT data (0.88). As SAP data is originally acquired using staircase procedures based on a logarithmic scale (dB), SAP is not good at estimating small amounts of ganglion cell losses at early stages of the disease. In contrast, by expanding the range of the scale at later stages, SAP might be more sensitive to small changes in the number of RGCs which do not seem to produce detectable changes in RNFL thickness. Despite these observations, the ability to express results of functional and structural tests in the same domain opens the possibility of combining the information from the two tests to increase the precision of RGC estimates, as performed in our study. By combining the estimates, one increases the precision of the final estimate of neuronal losses to better stage glaucomatous damage. However, instead of simply averaging the two estimates, we used a weighting scheme based on MD values. This was done in order to take into consideration differences in performance of SAP and imaging tests at different stages of the disease for the reasons described above.

The study has limitations. Empirically-derived formulas to estimate the number of RGCs from SAP and OCT data were used. Although estimates obtained from these formulas have been validated in multiple external cohorts, the original formula for estimating RGCs from OCT data was based on an older version of the technology, time-domain OCT. In our study, we used the same previously derived formulas, but data were obtained by SDOCT and it is possible that modifications would be necessary to compensate for the change in technologies. However, the agreement between SAP and OCT data found in our study was similar to that reported by Harwerth et al², suggesting that major modifications are probably not necessary. Another potential limitation of our study is that we used only global measures of visual function and structural damage. A sectorial analysis may provide a better representation of localized damage and improved detection of glaucoma. However, the use of sectorial information may be difficult to interpret in the context of a staging system. Additionally, sectorial information will be more variable and not necessarily better for monitoring changes over time. Further studies should evaluate whether a combination of sectorial structure and function data could improve detection and staging of glaucomatous damage. Another limitation of our study is that the presence of media opacities could potentially affect SAP-derived estimates of RGCs and, therefore, calculations of the CSFI. This is a potential limitation of most visual field-based staging systems, as they usually base their classifications at least in part on values of the MD index. However, by combining functional and structural measurements, our approach potentially reduces the effect of media opacities by relatively decreasing the influence of SAP-derived data on the final estimates of neuronal losses. Nevertheless, clinicians should be aware of the effect of media opacities when evaluating functional changes and quality of imaging test results in glaucoma patients.

The CSFI has several desirable properties for use as a staging index. It discriminates well among the different stages of the disease and has a very intuitive interpretation as the overall percent loss of neuronal tissue. In addition, it is provided on a continuous scale avoiding the artificial categorization of the disease continuum. However, it should be emphasized that an ideal staging system for glaucoma would be highly predictive of the degree of disability from the disease. Although SAP measurements have been related to measures of quality of vision in patients with glaucoma, such relationship is usually weak. Recent studies have proposed different methods to evaluate the degree of functional impairment caused by the disease and future studies should be performed attempting to correlate proposed staging systems to results of these tests or develop staging systems based on results of tests directly measuring functional impairment in glaucoma.^{34, 35} The methods described in our study to estimate RGC counts from a combination of structure and function could also be used to provide a useful parameter for longitudinal monitoring of glaucomatous changes. We are conducting additional studies to investigate this possibility.

In conclusion, an index combining structure and function performed better than isolated structural and functional measures for detection of perimetric and preperimetric glaucoma as well as for discriminating different stages of the disease. Further studies should evaluate the ability of the proposed index to monitor glaucomatous changes over time.

The imperfect relationship between structural and functional measurements of the disease seem to be largely derived from the different algorithms and measurement scales, as well as the different variability characteristics of the tests commonly used to assess structural and functional losses. In fact, Harwerth and colleagues⁴⁷ demonstrated that structural and functional tests are in agreement as long as one uses appropriate measurement scales for neural and sensitivity losses and considers factors such as the effect of aging and eccentricity on estimates of neural losses. In a series of investigations, they demonstrated that estimates of RGC losses obtained from clinical perimetry agreed closely with estimates of RGC losses obtained from RNFL assessment by optical coherence tomography (OCT).⁴⁷ The results of their model provided a common domain for expressing results of structural and functional tests, e.g., the estimates of RGC losses, opening the possibility of combining these different tests to improve the reliability and accuracy of estimates of the amount of neural losses in glaucoma.

In a second study, measurements of structural and functional tests were combined to provide an estimate of the rate of RGC loss in glaucoma patients followed up over time. We showed that the calculated estimates of the rate of RGC loss performed significantly better than isolated measures of structure or of function to detect disease progression over time.

A second observational study was performed as follows. Participants from this study were included in two prospective longitudinal studies designed to evaluate optic nerve structure and visual function in glaucoma (the African Descent and Glaucoma Evaluation Study [ADAGES] and the Diagnostic Innovations in Glaucoma Study [DIGS]). The 3-site ADAGES collaboration includes the Hamilton Glaucoma Center at the Department of Ophthalmology, University of California-San Diego (UCSD) (data coordinating center), the New York Eye and Ear Infirmary and the Department of Ophthalmology, University of Alabama, Birmingham (UAB). Although the DIGS includes only patients recruited at UCSD, the protocols of the two studies are identical. Methodological details have been described previously.⁴⁸

At each visit during follow-up, subjects underwent a comprehensive ophthalmologic examination including review of medical history, best-corrected visual acuity, slit-lamp biomicroscopy, intraocular pressure (IOP) measurement, gonioscopy, dilated fundoscopic examination, stereoscopic optic disc photography, and automated perimetry using Swedish Interactive Threshold Algorithm (SITA Standard 24-2). Only subjects with open angles on gonioscopy were included. Subjects were excluded if they presented with a best-corrected visual acuity less than 20/40, spherical refraction outside ±5.0 diopters and/or cylinder correction outside 3.0 diopters, or any other ocular or systemic disease that could affect the optic nerve or the visual field.

### Participants

The study included 3 groups of participants. The main study group was composed of 213 eyes of 213 glaucoma patients from the DIGS/ADAGES cohort followed for an average of 4.5±0.8 years. Eyes were classified as glaucomatous if they had evidence of glaucomatous optic neuropathy based on masked grading of optic disc stereophotographs and/or repeatable abnormal visual field test results on the baseline visit. Glaucomatous optic neuropathy was diagnosed based on the presence of neuroretinal rim thinning, excavation, or RNFL defects. Abnormal visual field was defined as a pattern standard deviation (PSD) outside of the 95% normal confidence limits, or a Glaucoma Hemifield Test result outside normal limits. All eyes were followed at approximately annual intervals with SAP and OCT testing and were required to have a minimum of 5 SAP and 5 OCTs during follow-up.

A control group of 33 eyes from 33 stable glaucoma patients was used to evaluate the specificity of our method. This set consisted of eyes with 5 serial visual fields and OCT exams collected under an IRB approved protocol within a maximum period of eight weeks from individuals seen at the Department of Ophthalmology, University of Miami Miller School of Medicine. All participating subjects were fully informed, and each signed a consent form. Each eye also had to have evidence of glaucoma at baseline based on ocular examination and the presence of repeated visual field loss as defined above. Mean MD and PSD values at the first visit were −7.4dB and 8.4dB. There was a wide range of disease severity in these eyes, with MD values ranging from −30.43dB to 0.91dB. The assumption was made that the disease was not progressing in these eyes over such a short time, and that any change noted would be due to the variability in the visual fields or OCT measurements in stable glaucoma. Therefore, the order of testing would be exchangeable and a permutation technique was used to provide a larger dataset to evaluate specificity. We generated all possible permutations of the order of the tests so that 3960 different sequences were obtained. For evaluation of rates of change in these eyes, the visits were annualized.

An additional group of 52 eyes from 52 healthy subjects followed for an average of 4.0±0.7 years was used to evaluate the effect of aging on the rate of RGC loss. All eyes were followed at approximately annual intervals with SAP and OCT testing and had an average of 4.4±0.6 tests acquired during follow-up. These subjects were recruited from the general population and were required to have a normal ophthalmologic examination, IOP below 22 mmHg in both eyes and normal visual field tests. Normal visual fields were defined as MD and PSD with P>0.05 and glaucoma hemifield test results within normal limits.

### Visual Field Testing

All patients underwent SAP testing using SITA-standard 24-2 strategy less than 6 months apart from imaging. All visual fields were evaluated by the UCSD Visual Field Assessment Center (VisFACT).⁴⁹ Visual fields with more than 33% fixation losses or false-negative errors, or more than 15% false-positive errors were excluded. The only exception was the inclusion of visual fields with false-negative errors of more than 33% when the field showed advanced disease (MD lower than −12dB).⁵⁰ Visual fields exhibiting a learning effect (e.g., initial tests showing consistent improvement on visual field indexes) were also excluded. Visual fields were further reviewed for the following artifacts: lid and rim artifacts, fatigue effects, inappropriate fixation, evidence that the visual field results were due to a disease other than glaucoma (such as homonymous hemianopia), and inattention. The VisFACT requested repeats of unreliable visual field test results, and these were obtained whenever possible.

### Optical Coherence Tomography

Subjects underwent ocular imaging with dilated pupils using the optical coherence tomograph StratusOCT™ (Carl Zeiss Meditec, Dublin, Calif.).⁵¹ Quality assessment of Stratus OCT scans was evaluated by an experienced examiner masked to the subject's results of the other tests. Good quality scans had to have focused images from the ocular fundus, signal strength greater than 7 and presence of a centered circular ring around the optic disc. The fast RNFL algorithm was used to obtain RNFL thickness measurements with Stratus OCT. Three images were acquired from each subject, with each image consisting of 256 A-scans along a 3.4 mm-diameter circular ring around the optic disc. The average parapapillary RNFL thickness (360° measure) was automatically calculated by the software and used in the study. RNFL scans were also evaluated as to the adequacy of the algorithm for detection of the RNFL. Only scans without overt algorithm failure in detecting the retinal borders were included in the study.

### Combined Structure and Function Estimate of RGC Counts

The development of the combined structure and function estimate of RGC counts was based on previous work by Harwerth and colleagues⁴⁷ on the development and validation of a model linking structure and function in glaucoma^{39,47}. Based on experimental studies in monkeys, the authors first derived an empirical model relating sensitivity measurements in SAP to histological RGC counts as a function of retinal eccentricities. The experimental results were then translated to clinical perimetry in humans. The following formulas were proposed to estimate the number of RGC somas in an area of the retina corresponding to a specific SAP test field location at eccentricity ec with sensitivity s in dB:

m=[0.054*(ec*1.32)]+0.9

b=[−1.5*(ec*1.32)]−14.8

gc={[(s−1)−b]/m}+4.7

SAPrgc=Σ10̂(gc*0.1)

In the above formulas, m and b represent the slope and intercept, respectively, of the linear function relating ganglion cell quantity (gc) in decibels to the visual field sensitivity (s) in decibels at a given eccentricity. To account for the total number of ganglion cells in an area of the retina, the cell density derived from each perimetry measurement was considered to be uniform over an area of retina corresponding to an area of 6×6 degrees of visual space that separates test locations in SAP. By applying the above formulas, a SAP-derived estimate of the total number of RGCs (SAPrgc) was obtained by adding the estimates from all locations in the visual field. The structural part of the model consisted in estimating the number of RGC axons from RNFL thickness measurements obtained by optical coherence tomography. The model took into account the effect of aging in the axonal density and the effect of disease severity on the relationship between the neuronal and non-neuronal components of the RNFL thickness estimates obtained by OCT. To derive the total number of RGC axons from the global RNFL thickness measurement obtained by OCT (OCTrgc), we applied the following formulas:

d=(−0.007*age)+1.4

c=(−0.26*MD)+0.12

a=average RNFL thickness*10870*d

OCTrgc=10̂[(log(a)*10−c)*0.1]

In the above formulas, d corresponds to the axonal density (axons/μm²), and c is a correction factor for the severity of disease to take into account remodeling of the RNFL axonal and nonaxonal composition. The average RNFL thickness corresponds to the 360-degree measure automatically calculated by the OCT software. These calculations provide an estimate of the number of RGCs from two sources, one functional and one structural, and a strong relationship was demonstrated between the two estimates in external validation cohorts.⁴⁷ However, although Harwerth et al⁴⁷ proposed a model linking structure and function, no attempt was made to obtain a combined estimate derived from structural and functional tests that could be clinically used to stage glaucoma severity and detect change over time. We developed such a combined measure by averaging the estimates of RGC numbers obtained from SAP and from OCT, but weighting according to severity of disease. Because clinical perimetry and imaging tests accuracies are inversely related to disease severity, we used a weighted scale that combined the estimates of RGC numbers from both tests:

Combined RGC count=(1+MD/30)*OCTrgc+(−MD/30)*SAPrgc

The weights were chosen to reflect the inverse relationship with disease severity of SAP and OCT estimates, along the scale of MD values ranging from 0 to −30dB. Therefore, in early disease, the OCT-derived RGC estimates will have greater weight than those obtained by SAP. In contrast, in advanced disease, SAP estimates will carry greater weight than those obtained from OCT.

After the combined estimates of RGC number were obtained, a linear mixed effects model was run to evaluate the effect of aging on RGC loss in the 52 healthy eyes followed longitudinally.⁵² The purpose was to calculate the effect of normal aging on the rate of RGC loss so that glaucomatous progression would be considered to occur if the rate of RGC loss was greater than the expected age-related loss. The linear mixed effects model showed a significant effect of age on the number of RGCs over time with a loss of 7877 RGCs per 1 year older (P<0.001). For each eye, we obtained the slope of change using ordinary least squares (OLS) linear regression of the combined RGC counts over time. An eye was considered to have progressed if the slope of RGC loss was significantly faster than the age-expected decline of RGC counts with P<0.05.

Slopes were also calculated for the raw values of OCT average thickness and for the SAP visual field index (VFI) provided by the Humphrey perimeter (Carl-Zeiss Meditec, Inc., Dublin, Calif.).⁵³ The VFI represents the percent of normal age-corrected visual function and is the method currently used for calculating rates of progression in the Humphrey visual field printout. Details of the calculation of the VFI have been described elsewhere.⁵³ The VFI can range from 100% (normal visual field) to 0% (perimetrically blind field). Progression by OCT average thickness or by VFI was defined based on the presence of a statistically significant negative slope with P<0.05.

All statistical analyses were performed with commercially available software (Stata version 12; StataCorp, College Station, Tex.). Cluster-correlated robust estimates of variance were used to adjust for correlated data when necessary.⁵⁴ The alpha level (type I error) was set at 0.05.

### Results

The main study group was composed of 213 eyes with mean age of 60±11 years at baseline. Average MD and PSD values of the baseline visual field test were −2.51dB and 3.34dB. Average baseline RNFL thickness was 88 μm (±15 μm). These eyes had a wide range of disease seventies at baseline with MD values ranging from −20.1dB to 2.14dB. A median number of 5 pairs of SAP and OCT tests were available during follow-up for these eyes, ranging from 5 to 8.

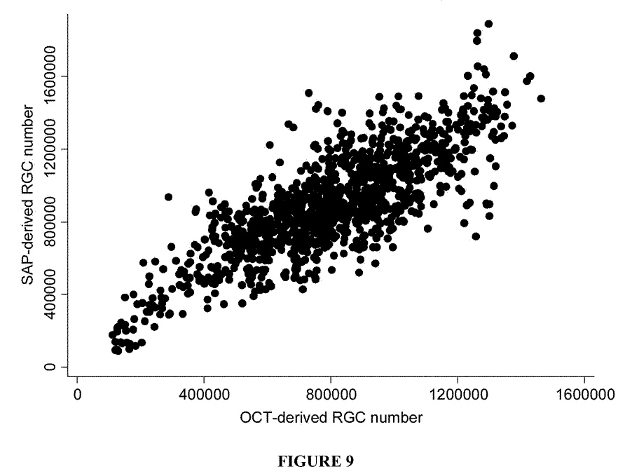

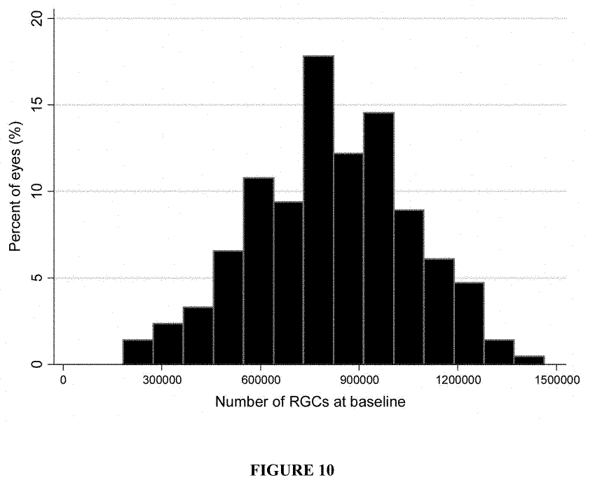

There was a strong correlation between RGC estimates obtained from SAP and OCT data for all exams from the 213 eyes included in the study group (r=0.80; P<0.001) (FIG. 9). FIG. 10 shows a histogram of calculated RGC numbers combining structural and functional tests at the baseline visit for these eyes. The mean number of RGCs was 765745 (±270029) at baseline which was significantly lower than the mean number of RGCs in the 52 healthy eyes (1123504±172667; P<0.001).

From the 213 eyes, 47 (22.1%) showed statistically significant rates of RGC loss that were faster than the age-expected decline. The mean rate of RGC loss in these eyes was −33369 cells/year (range: −8332 cells/year to −80636 cells/year). There was no statistically significant difference between mean baseline RGC counts for progressing versus non-progressing eyes (797229 vs. 758527; P=0.377). We estimated a percent rate of RGC loss by dividing the calculated rate of RGC loss by the baseline RGC count. The mean percent rate of RGC loss was −4.4%/year for the 47 progressing eyes, ranging from −1.4%/year to −8.9%/year.

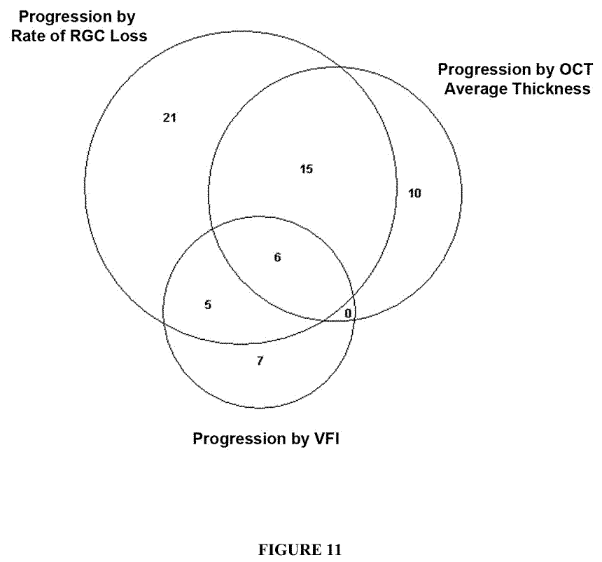

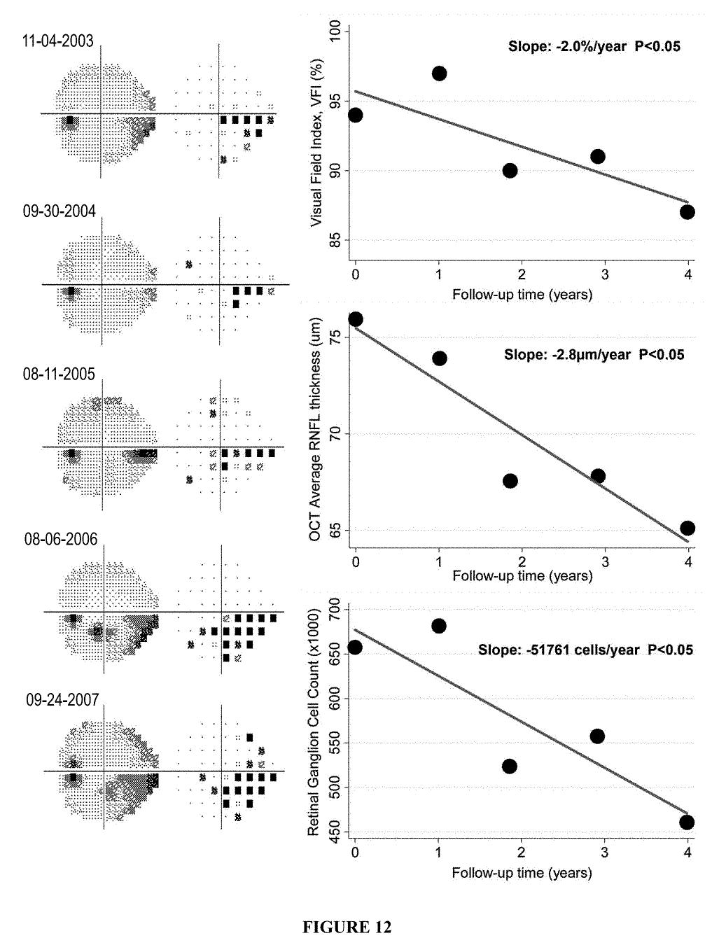

The VFI was able to detect progression in only 18 (8.5%) of the 213 eyes whereas the OCT parameter average RNFL thickness detected progression in 31 eyes (14.6%). FIG. 11 shows a proportional Venn diagram with the number of eyes detected as progressing by each method. FIG. 12 shows an example of an eye with significant rate of RGC loss which also progressed by VFI and OCT average thickness.

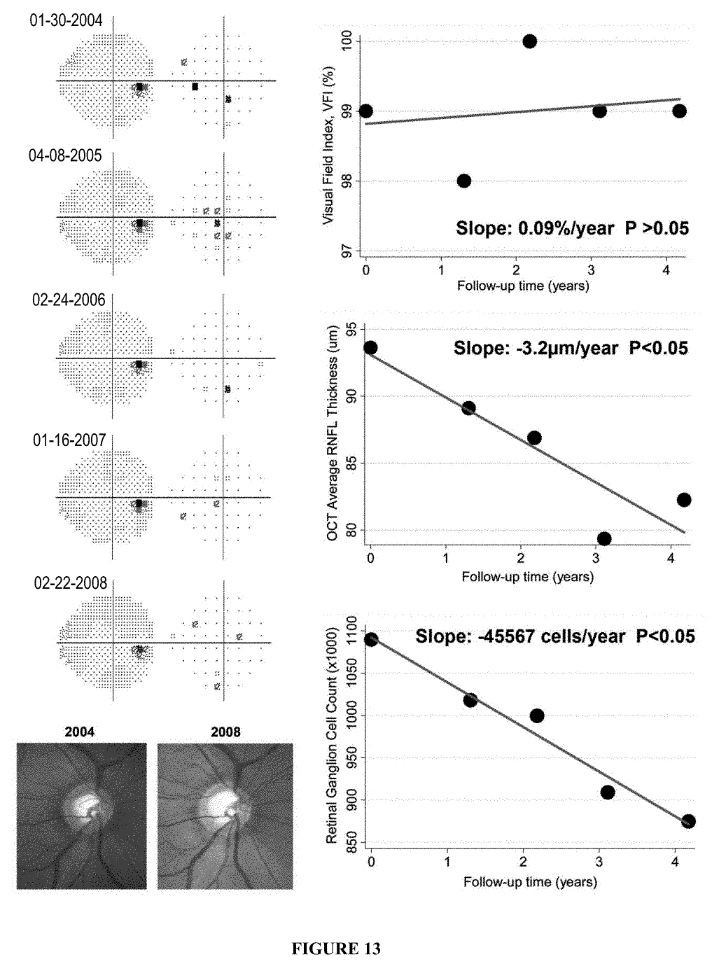

Thirty-six eyes had progression detected by the rate of RGC loss but not by the VFI. These eyes had a mean rate of RGC loss of −32310 cells/year. Seven eyes had progression detected by the VFI but not by the rate of RGC loss. These eyes had a rate of RGC loss of only −3393 cells/year. A comparison between these two groups also revealed that eyes progressing only by the rate of RGC loss had significantly faster rates of structural change than those progressing only by VFI as measured by OCT average RNFL thickness (−1.89μm/year versus 0.37μm/year, respectively; P=0.002). FIG. 13 shows an example of an eye detected as progressing according to the estimated rate of RGC loss but not by the VFI.

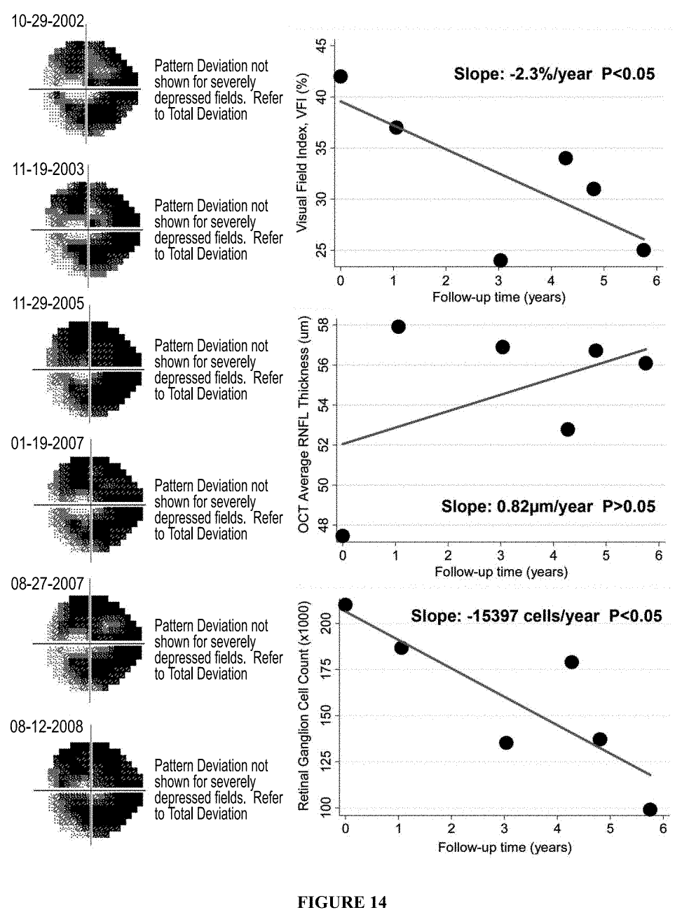

Twenty-six eyes had progression detected by the rate of RGC loss but not by OCT average thickness, whereas 10 eyes had progression detected by OCT average thickness but not by the rate of RGC loss. The former group had a mean rate of RGC loss of −32486 cells/year versus −7539 cells/year in the latter. A comparison between these two groups also revealed that eyes progressing only by the rate of RGC loss had significantly faster rates of functional change than those progressing only by OCT as measured by the VFI (−0.65%/year versus 0.55%/year; P=0.003). FIG. 14 shows an example of an eye detected as progressing according to the estimated rate of RGC loss but not by the OCT parameter average thickness.

### Evaluation of Specificity

Specificity for detection of change was evaluated in the 3960 sequences of tests generated from the 33 eyes of the stable data. The rate of RGC loss was statistically significant in 203 (5%) of the 3960 sequences resulting in specificity of 95%. The OCT parameter average thickness detected change in 199 sequences (specificity of 95%) and the VFI detected change in 174 sequences (specificity of 96%).

The proportions of eyes from the main study group that were detected as progressing by each method at the matched specificities was compared. The proportion progressing by rates of RGC loss was larger than that progressing only by OCT average thickness (22.1% vs. 14.6%; P=0.01) and by VFI (22.1% vs. 8.5%; P<0.001).

### Discussion

In the second study, the evaluation of rates of neuronal loss based on estimates of RGC counts combining structure and function was demonstrated to be able to detect a larger number of glaucomatous eyes as progressing compared to the use of isolated measures of SAP or OCT, while maintaining comparable specificity in a group of stable eyes. To our knowledge this is the first study to develop and evaluate the ability of a single measure of RGC count combining structure and function for detection of glaucoma progression.

Several studies have shown that considerable disagreement is present when different structural and functional tests are used to detect disease progression.^{42-44,46,55,56} More specifically, SAP seems to be relatively insensitive to detect change in early stages of the disease, whereas structural assessment by imaging instruments seem to perform relatively worse at advanced stages of damage. The disagreement between structure and function, however, seems to be largely derived from the different algorithms and measurement scales of the tests commonly used to assess losses. In fact, Harwerth and colleagues⁴⁷demonstrated a strong agreement between structural and functional tests when appropriate measurement scales for neural and sensitivity losses were used. Our present results agree with those previously published by Harwerth et al, as shown by the strong linear relationship between RGC estimates obtained from SAP and OCT data. The linear relationship suggests that the lack of sensitivity of SAP for detection of progression in early disease is most likely not the result of true structural changes occurring in the absence of functional losses, but is rather related to the logarithmic scale used for SAP sensitivity measurements. Such result has also been suggested by other authors.^{38,57} The logarithmic scale compresses the range of losses in early stages of the disease while expanding the range in later stages. In principle, this could suggest that a simple linearization of SAP data could improve detection of early losses. However, this is usually not the case.⁵⁸ As SAP data is originally acquired using staircase procedures based on a logarithmic scale (dB), SAP is not good at estimating small amounts of ganglion cell losses at early stages of the disease. In contrast, by expanding the range of the scale at later stages, SAP might be more sensitive to small changes in the number of RGCs which do not seem to produce detectable changes in RNFL thickness. This highlights the need for a combined approach using structure and function to detect disease progression.⁵⁹⁻⁶¹ The ability to express results of functional and structural tests in the same domain opens the possibility of combining the information from the two tests to increase the precision of RGC estimates, as performed in our study. By combining the estimates, one increases the precision of the final estimate of neuronal losses to better detect change over time. However, instead of simply averaging the two estimates, we used a weighting scheme based on MD values. This was done in order to take into consideration differences in performance of SAP and imaging tests at different stages of the disease for the reasons described above.

Our estimates of RGC losses detected a significantly larger number of glaucomatous eyes as progressing compared to isolated measures of structure and of function, despite having the same specificity in the stable data. It detected the majority of eyes progressing by VFI or OCT. However, some disagreement was seen among the different methods as seen on FIG. 11. Interestingly, 36 eyes had progression detected by rates of RGC loss but not by the VFI, whereas 7 eyes had progression detected by the VFI but not by the rate of RGC loss. A comparison between these two groups revealed that eyes progressing only by rates of RGC loss had concomitant evidence of structural change, whereas in eyes progressing only by VFI no such evidence was present. It should be noted that at 95% specificity, approximately 10 of the 213 eyes would be expected to show significant slopes just by chance. In the absence of supportive concomitant structural changes, it is likely that the 7 eyes showing progression by the VFI but not by rates of RGC loss could represent just false positives. Similarly, eyes progressing only based on the rates of RGC loss had significantly faster rates of functional change than those progressing only by OCT as measured by the VFI. The presence of concomitant structural and functional change in eyes progressing by rates of RGC loss provides stronger support suggesting that these eyes represented true progressors compared to those progressing only by VFI or by OCT. Twenty-one eyes had progression by rates of RGC loss but neither by VFI nor by OCT average thickness. These eyes had a mean rate of RGC loss of −31009 cells/year. The mean rates of VFI and OCT average thickness change were −0.51%/year and −0.98μm/year, respectively. It is likely that the amount of change in these eyes was not enough to declare progression based only on the results of the structural or the functional test. However, the combination of measurements from both tests allowed detection of significant change in these eyes. It is also important to note that no eye was detected as progressing by VFI and OCT average thickness, but not by the calculated rate of RGC loss, as shown on FIG. 11.

Clinicians are frequently faced with the task of integrating results from structural and functional testing to detect glaucoma progression. This is done routinely as they attempt to correlate changes in their examinations of the optic nerve to those occurring in the visual field, so that if changes over time are seen in both methods, they are more reassuring to indicate true deterioration. However, clinicians are frequently uncertain about how to interpret apparently conflicting results coming from different tests. Also, the use of many different tests can increase the chance of a type I error, e.g., declaring as significant a change that actually has occurred by chance. In fact, if we had declared progression based on the presence of significant change on either SAP, VFI or OCT average thickness, the specificity in the stable dataset would have decreased to 90.7%. That is, from the 213 eyes, approximately 20 eyes would be expected to be false positives. By providing a single index of RGC loss combining structural and functional information, we are able to better control type I error. In fact, by setting the alpha to 0.05 to declare the slope of RGC loss as statistically significant, we were able to maintain a specificity of 95%, as demonstrated in the stable group. In addition, we also required that the slopes of RGC loss had to be faster than the age-expected RGC losses for an eye to be considered progressing. This may also represent an additional advantage of our method compared to detection of change based on raw indexes such as OCT average thickness, for example, especially when a large series of tests is being evaluated over a long time period.

An ideal method for detection of glaucomatous progression should not only give an indication of whether the eye or the patient is likely showing progression, but also needs to give an estimate of the rate of deterioration. Although most glaucoma patients will show some evidence of progression if followed long enough, the rate of deterioration can be highly variable among them.^{45,62-65} While most patients progress relatively slowly, others have aggressive disease with fast deterioration which can eventually result in blindness or substantial impairment unless appropriate interventions take place. The proposed index allows estimation of the rate of RGC loss over time from structural and functional measurements and has an intuitive meaning which should facilitate the interpretation of rates of change by clinicians. From the 47 eyes detected as progressing by rates of RGC loss, 14 (30%) had rates faster than −5%/year. In principle, these eyes could be considered fast progressors, as their rate of progression would result in 50% loss of their RGCs from the baseline value in a 10-year period. It is important to emphasize, however, that when assessing the clinical relevance of an estimated rate of RGC loss, clinicians also need to consider other factors, such as life expectancy and the patient's expectations with regard to treatment.

The VFI was used to evaluate rates of visual field loss using SAP. This index has been incorporated into the Guided Progression Analysis software and is the current method used to analyze rates of visual field loss with the Humphrey perimeters. A recent study, however, has suggested that the reliance of the VFI on pattern deviation probability maps may cause a ceiling effect that may reduce its sensitivity to change in eyes with early damage.⁶⁶ Therefore, we also analyzed rates of visual field loss using the parameter MD. For a specificity of 95% in the stable group, only 16 (7.5%) of the 213 eyes had progression based on rates of MD change, a number significantly lower than that found using combined estimates of RGC loss (P<0.001).

Structural and functional measurements for detection of glaucoma progression using Bayesian methodology have previously been combined.⁵⁹ The Bayesian approach provided an effective method of combining results of different tests to improve estimates of rate of progression and also incorporate risk factors for detection of change. Compared to the Bayesian method, the current approach has the potential advantage of using a single estimate of RGC counts obtained from structural and functional tests which potentially facilitates clinical interpretation. However, the Bayesian approach provides the flexibility of combining multiple different tests including structural measurements derived by other imaging technologies such as confocal scanning laser ophthalmoscopy or scanning laser polarimetry and function-specific perimetric tests. Although the principles outlined in our study could in theory be applied to these other tests, the specific methods for translating measurements to RGC counts have not yet been established. It should be noted, however, that a combination of the two methodologies should be possible, such as incorporating risk factors to improve estimation of rates of RGC loss, but the benefits of such approach would have to be evaluated on an independent sample of patients.

Our study has limitations. Empirically derived formulas were used to estimate the number of RGCs from SAP and OCT data. Although these estimates have been validated in histologic studies in monkeys and also have been applied to multiple external cohorts in humans,⁴⁷ such validation was not based on direct histologic RGC counts in humans. However, this limitation applies to most measurements obtained in clinical practice from imaging devices and other instruments. This study clearly showed a benefit of our method in detecting glaucoma progression, and even though a full histologic validation is not available at this time, this should not preclude its usefulness in clinical practice. It is interesting to note that despite absence of histologic validation, the age-related loss of RGCs (7877 RGCs per year) found in our study was very similar to that found in previous histologic studies in humans.⁶⁷ It is possible that other weighting schemes for combination of SAP and OCT estimates of RGC counts could perform better than the one proposed in our study. When an analysis was performed using a simple average of RGC counts from SAP and OCT without weighting, the method detected progression in only 28 eyes compared with 47 eyes for our proposed weighting scheme, at similar specificities. When the weighting system was based on antilog MD values, the performance also was inferior, detecting only 28 eyes as progressing for similar specificity. Further studies should evaluate other methods of combining SAP and OCT estimates of RGC loss and should test them on independent populations. In addition, further developments in perimetry and imaging techniques potentially may improve estimates of RGC counts obtained by these instruments, leading to improved detection of change.⁶⁸

We used OCT measurements based on the time-domain version of this technology. The use of spectral-domain OCT (SDOCT) has resulted in faster and more reproducible scans compared to time-domain OCT.⁶⁹ In a previous cross-sectional study, we developed a combined index of RGC count which used SDOCT measurements along with SAP results. The index performed better than isolated measures of structure and function to stage disease severity.⁵⁸ However, due to the relatively recent introduction of SDOCT, longitudinal data was not available to perform the current study using this technology. Another potential limitation of our study is that we used only global measures of visual function and structural damage. A sectorial analysis may provide a better representation of localized damage and improved detection of progression. However, sectorial information will be more variable and not necessarily better for monitoring changes over time. Further studies should evaluate whether a combination of sectorial structure and function data could improve detection of glaucomatous change.

In conclusion, an index estimating the rate of RGC loss combining structure and function performed better than isolated structural and functional measures for detecting progressive glaucomatous damage. The use of such index may improve detection of change in clinical practice and in trials evaluating disease progression.

The goal of glaucoma management is to slow down the rate of progressive neural losses in order to preserve visual function during the patient's lifetime. Assessment of visual function in clinical practice is traditionally performed with standard automated perimetry (SAP). However, although SAP testing has been widely used for diagnosis, staging and monitoring the disease, it has become increasingly evident that a substantial number of RGCs may need to be lost before damage to SAP becomes statistically significant.⁷⁰⁻⁷⁹

In a study of cadaver eyes of patients with glaucoma who had previously undergone SAP, Kerrigan-Baumrind et al.⁸⁰ estimated that at least 25% to 35% of RGCs would need to be lost for statistically significant abnormalities to appear on automated perimetry. However, these estimates were based on a relatively small number of eyes, and no follow-up data were available to determine precisely when visual field defects first occurred. Although direct RGC counting in vivo is not yet possible in humans, the use of empirical formulas derived from clinical structural and functional tests may give estimates of the number of RGCs that have been shown to correlate well with histologic counts in experimental glaucoma models.^{81,82} In recent studies, we proposed a method for estimating the amount of RGC losses from a combination of retinal nerve fiber layer (RNFL) assessment with optical coherence tomography (OCT) and SAP.⁸³⁻⁸⁵ The estimates of RGC counts performed significantly better than isolated structural and functional parameters for staging the disease and monitoring glaucomatous progression.

In this study, we provided estimates of RGC losses associated with the earliest development of visual field defects in glaucoma. To assess RGC losses at this stage of the disease, a cohort of patients with suspected glaucoma was followed until initial development of repeatable and statistically significant visual field defects on SAP. By using this approach, we were able to quantify the magnitude of estimated RGC losses associated with the development of significant SAP abnormalities from the disease.

A third observational study was performed as follows. Participants from this study were included in 2 prospective longitudinal studies designed to evaluate optic nerve structure and visual function in glaucoma: the Diagnostic Innovations in Glaucoma Study (DIGS) and the African Descent and Glaucoma Evaluation Study (ADAGES). The 3-site ADAGES collaboration includes the Hamilton Glaucoma Center at the Department of Ophthalmology; the University of California-San Diego (UCSD) (data coordinating center); the New York Eye and Ear Infirmary; and the Department of Ophthalmology, University of Alabama, Birmingham. Although the DIGS includes only patients recruited at the UCSD, the protocols of the two studies are identical. The institutional review boards at all 3 sites approved the study methodology, which adhered to the tenets of the Declaration of Helsinki and to the Health Insurance Portability and Accountability Act. Methodological details have been described previously.⁸⁶

At each visit during follow-up, subjects underwent a comprehensive ophthalmologic examination including review of medical history, best-corrected visual acuity, slit-lamp biomicroscopy, intraocular pressure measurement, gonioscopy, dilated fundoscopic examination, stereoscopic optic disc photography, and automated perimetry using the Swedish Interactive Threshold Algorithm (Standard 24-2). Only subjects with open angles on gonioscopy were included. Subjects were excluded if they presented with a best-corrected visual acuity less than 20/40, spherical refraction outside ±5.0 diopters or cylinder correction outside 3.0 diopters, or any other ocular or systemic disease that could affect the optic nerve or visual field.

### Participants

The study group consisted of 53 eyes of 53 patients with suspected glaucoma who were followed as part of the DIGS/ADAGES cohort and developed repeatable abnormal visual fields during follow-up, that is, converted to glaucoma. Initial diagnosis as suspected glaucoma was based on the presence of suspicious appearance of the optic disc or elevated (>21 mmHg) intraocular pressure, but normal SAP testing at baseline. Normal visual fields were defined on the basis of mean deviation (MD) and pattern standard deviation (PSD) within 95% confidence limits and a Glaucoma Hemifield Test within normal limits. These eyes had a median follow-up of 6.7 years (first quartile: 4.4 years, third quartile: 13.3 years) until the development of repeatable abnormal SAP defects. Repeatable abnormal SAP was defined on the basis of the presence of a sequence of three consecutive abnormal SAPs with PSD with P<5% or Glaucoma Hemifield Test outside normal limits. Imaging assessment of the RNFL with spectral domain OCT (SD-OCT) was performed at the time (within ±3 months) of the first visual field of the sequence of three repeatable abnormal fields. This was performed to calculate estimates of RGC counts (see “Estimates of Retinal Ganglion Cell Counts”) at the time of detection of the earliest visual field defect on SAP.

An age-matched control group consisting of 124 eyes from 124 healthy participants was included in the study. These subjects were recruited from the general population and were required to have normal ophthalmologic examination results and an intraocular pressure <22 mmHg in both eyes, but results of visual field tests and SD-OCT were not used as inclusion or exclusion criteria. Healthy eyes were chosen as the control group because we were interested in evaluating the amount of RGC loss associated with early visual field defects compared with normal expected age-matched RGC counts. Although a group of glaucoma suspects who did not develop visual field loss could be initially thought of as a control group, these eyes could have sustained structural damage before functional losses and therefore would not constitute a suitable control group for the purposes of this study.

### Visual Field Testing

All patients underwent SAP testing using the Swedish Interactive Threshold Algorithm Standard 24-2 strategy during follow-up. All visual fields were evaluated by the UCSD Visual Field Assessment Center.⁸⁷ Visual fields with more than 33% fixation losses or false-negative errors or more than 15% false-positive errors were excluded. Visual fields exhibiting a learning effect (i.e., initial tests showing consistent improvement on visual field indexes) also were excluded. Visual fields were further reviewed for the following artifacts: lid and rim artifacts, fatigue effects, inappropriate fixation, evidence that the visual field results were due to a disease other than glaucoma (e.g., homonymous hemianopia), and inattention. The UCSD Visual Field Assessment Center requested repeats of unreliable visual field test results, and these were obtained whenever possible.

### Spectral Domain Optical Coherence Tomography

The Cirrus HDOCT (software v. 5.2, Carl Zeiss Meditec Inc., Dublin, Calif.) was used to acquire RNFL measurements in the study. It uses a superluminescent diode scan with a center wavelength of 840 nm and an acquisition rate of 27 000 A-scans per second at an axial resolution of 5 μm. The protocol used for RNFL thickness evaluation was the optic disc cube. This protocol is based on a 3-dimensional scan of a 6×6 mm² area centered on the optic disc where information from a 1024 (depth)×200×200-point parallelepiped is collected. Then, a 3.46-mm-diameter circular scan (10 870 μm in length) is automatically placed around the optic disc, and the information about parapapillary RNFL thickness is obtained. Because information from the whole region is obtained, it is possible to modify the position of the scan after the examination is taken. To be included, all images were reviewed for noncentered scans and had to have a signal strength >6, absence of movement artifacts, and good centering on the optic disc. For estimation of overall RGC counts, we used the parameter average RNFL thickness (360-degree measure around the optic disc). For estimation of RGC counts on each hemiretina, we calculated the average RNFL thickness at each semicircle of 180 degrees around the optic disc.

### Estimates of Retinal Ganglion Cell Counts

The estimates of RGC counts were obtained according to the model developed by Medeiros et al^{83,84} based on empirical formulas derived by Harwerth et al⁸² for estimating ganglion cell counts from SAP and OCT. The model uses information from structural and functional tests to derive a final estimate of the RGC count in a particular eye. The details of the model and the empirical formulas used to derive RGC counts have been described in detail in previous publications.^{83,84} The initial step of the model consists in translating SAP sensitivity values into RGC counts using empirical formulas derived by experimental research in monkeys and subsequently translated to normal and glaucomatous human eyes.^{73,82} The following formulas were used to estimate the number of RGC somas in an area of the retina corresponding to a specific SAP test field location at eccentricity ec with sensitivity s in decibels:

m=[0.054*(ec*1.32)]+0.9

b=[−1.5*(ec*1.32)]−14.8

gc={[(s−1)−b]/m}+4.7

SAPrgc=Σ10̂(gc*0.1)

In these formulas, m and b represent the slope and intercept, respectively, of the linear function relating ganglion cell quantity (gc) in decibels to the visual field sensitivity (s) in decibels at a given eccentricity. To account for the total number of ganglion cells in an area of the retina, the cell density derived from each perimetry measurement was considered to be uniform over an area of retina corresponding to an area of 6×6 degrees of visual space that separates test locations in SAP. By applying the above formulas, a SAP-derived estimate of the total number of RGCs (SAPrgc) was obtained by adding the estimates from all locations in the visual field. The structural part of the model consisted in estimating the number of RGC axons from RNFL thickness measurements obtained by OCT. The model took into account the effect of aging in the axonal density and the effect of disease severity on the relationship between the neuronal and nonneuronal components of the RNFL thickness estimates obtained by OCT. To derive the total number of RGC axons from the global RNFL thickness measurement obtained by OCT (OCTrgc), we applied the following formulas:

d=(−0.007*age)+1.4

c=(−0.26*MD)+0.12

a=average RNFL thickness*10870*d

OCTrgc=10̂[(log(a)*10−c)*0.1]

In these formulas, d corresponds to the axonal density (axons μm²) and c is a correction factor for the severity of disease to take into account remodeling of the RNFL axonal and nonaxonal composition. These calculations provide an estimate of the number of RGCs from 2 sources, one functional and one structural. A combined calculation of RGC counts was performed according to the following formula:

RGC count=(1+MD/30)*OCTrgc+(−MD/30)*SArgc

The rationale for using a weighting system for deriving the final RGC count is described by Medeiros et al,⁸³⁻⁸⁵ but in essence it relies on the fact that the accuracies of clinical perimetry and imaging tests are inversely related to disease severity.

RGC counts were also obtained separately for each hemifield of the retina, using corresponding visual field sensitivities and RNFL thickness measurements.

### Statistical Analysis

Descriptive statistics included mean and standard deviation for normally distributed variables, and median, first quartile, and third quartile values for nonnormally distributed variables. Student t tests or Mann-Whitney U tests were used to evaluate demographic and clinical differences between glaucoma and control subjects in each of the analyses.

The performance of the RGC counts to discriminate glaucomatous eyes with early visual field defects from healthy eyes was compared with that of standard SD-OCT parameters. No comparison was performed against visual field parameters because these were used in the definition of the glaucoma group. Receiver operating characteristic (ROC) curves were built, and the area under the ROC curve was used to summarize the diagnostic accuracy for each parameter. An ROC curve area equal to 1 represents perfect discrimination, whereas an area of 0.5 represents chance discrimination. The ROC curve areas and 95% confidence intervals were obtained for each parameter after adjusting for age, using a previously described method.^{88,89} Evaluation of diagnostic accuracy also was performed using likelihood ratios (LRs). The LR is defined as the probability of a given test result in those with disease divided by the probability of the same test result in those without disease.^{90,91} Once determined, an LR can be directly incorporated into the calculation of posttest probability of disease by using a formulation of the Bayes' theorem.⁹² The LR for a given test result indicates how much that result will increase or decrease the pretest odds of disease. Application of LRs in the interpretation of results of imaging instruments for glaucoma diagnosis has been detailed elsewhere.^{93,94} A value of 1 means that the test provides no addition information, and ratios more or less than 1 increase or decrease the likelihood of disease, respectively.

All statistical analyses were performed with commercially available software (Stata version 12; StataCorp, College Station, Tex.). The alpha level (type I error) was set at 0.05.

### Results

There were 53 eyes of 53 subjects who developed visual field loss during follow-up and were included in the glaucoma group. At the baseline visit, average MD and PSD for these eyes were −0.98±1.39 dB and 1.96±0.56 dB, respectively. Corresponding values were −2.17±1.34 dB and 2.48±0.44 dB, respectively, at the time of the first abnormal visual field of the conversion sequence, that is, at the time of estimation of RGC counts. The average age at the time of conversion was 69±12 years. This group was compared with 124 eyes of 124 healthy subjects with an average age of 66±11 years. There was no statistically significant difference in mean age between the 2 groups (P=0.07). Average MD and PSD values for the healthy eyes were 0.11±1.23 dB and 1.67±0.59 dB, respectively. Table 1 summarizes the clinical and demographic parameters in the glaucoma and control groups.

TABLE 1
Clinical and Demographic Variables in the Glaucoma
and Healthy Groups

| | Glaucoma | Healthy | |
|---|---|---|---|
| | (n = 53) | (n = 124) | P |
| Age (yrs) | 69 ± 12 | 66 ± 11 | 0.07 |
| Race | | | |
| Caucasians | 37 | 94 | 0.41 |
| African-Americans | 16 | 30 | |
| Sex, female | 33 (62%) | 85 (69%) | 0.42 |
| MD* | −2.17 ± 1.34 | 0.11 ± 1.23 | <0.001 |
| PSD* | 2.48 ± 0.44 | 1.67 ± 0.59 | <0.001 |
| Average RNFL | 76.0 ± 9.9 | 91.6 ± 8.9 | <0.001 |
| thickness | | | |
| Estimated RGC | 652.057 ± 115.829 | 910.584 ± | <0.001 |
| count | | 142.412 | |

MD—mean deviation;
PSD—pattern standard deviation;
ROC—retinal ganglion cell;
RNFL—retinal nerve fiber layer.
VFI—visual field index;
*MD and PSD for glaucomatous eyes correspond to the values obtained from the first abnormal visual field of the conversion sequence. Values correspond to mean ± standard deviation, unless specified otherwise.

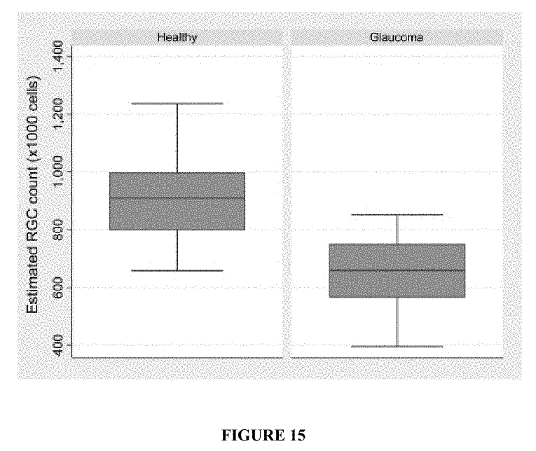

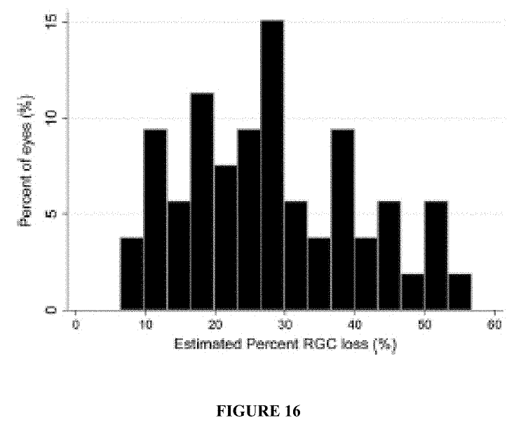

The average RGC count estimate in the eyes with early visual field defects was 652 057±115 829 cells, which was significantly lower than the average of 910 584±142 412 cells found in healthy eyes (P<0.001). FIG. 15 illustrates the distribution of RGC estimates in the glaucoma and control groups. Compared with the average number of RGCs in the healthy group, glaucomatous eyes had an average RGC loss of 28.4% (95% confidence interval, 24.9-31.9), ranging from 6% to 57%. FIG. 16 illustrates the distribution of percent RGC losses in the glaucoma group.

Twenty-two of the 53 glaucomatous eyes (42%) developed superior visual field defects, 14 eyes (26%) developed inferior defects, and 17 eyes (32%) had defects both superiorly and inferiorly. For the 22 eyes with superior visual field defects, RGC counts corresponding to the inferior hemiretina were significantly lower than those from the superior hemiretina (283 341±55 526 vs. 340 931±63 888, respectively; P<0.001). For the 14 eyes with inferior defects, RGC counts from the superior hemiretina were significantly lower than those from the inferior hemiretina (303 964±56 160 vs. 360 191±75 103, respectively; P<0.001). For the 17 eyes with defects both superiorly and inferiorly, there was no statistically significant difference between RGC counts in the superior and inferior hemiretinas (343 849±58 424 vs. 329 762±56 306, respectively; P=0.12). For the 124 healthy eyes, there was no significant difference between RGC counts in the superior and inferior hemiretinas (459 557±75 292 vs. 451 447±76 208, respectively; P=0.052).

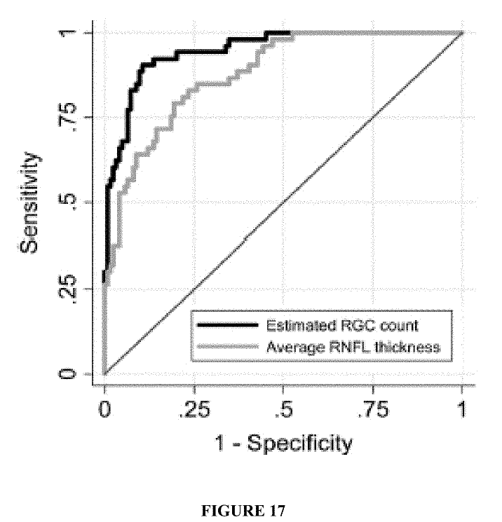

The RGC counts performed significantly better than the SD-OCT average RNFL thickness parameter in discriminating glaucomatous from healthy eyes, with ROC curve areas of 0.95±0.02 and 0.88±0.03, respectively (P=0.001) (FIG. 17). For 95% specificity, RGC counts had a sensitivity of 68% for detection of early glaucomatous damage with a positive LR of 13.6, whereas SD-OCT average RNFL thickness had a sensitivity of 53% with a positive LR of 10.6. For 90% specificity, sensitivity of RGC counts increased to 89% versus 64% for SD-OCT average RNFL thickness.

### Case Examples

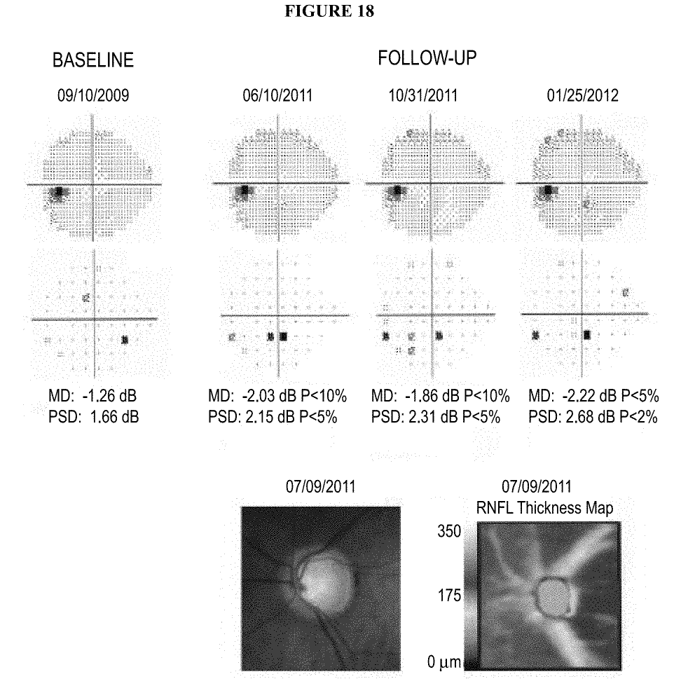

FIG. 18 illustrates an eye that had an estimated RGC count of 520 950 cells at the time of development of the initial visual field defect on SAP, corresponding to a 43% RGC loss compared with the healthy group. The defect was confirmed on subsequent tests based on the criterion of 3 consecutive abnormal fields with PSD, with P<5%. The optic disc photograph shows extensive neuroretinal rim loss in agreement with the RNFL loss assessed by SD-OCT, which showed an average RNFL thickness of 58 μm. Despite the extensive RGC loss, the visual field defect on the pattern deviation plot was apparently small with only an inferior cluster of abnormal points, although there was evidence of diffuse loss of sensitivity as indicated by the MD.

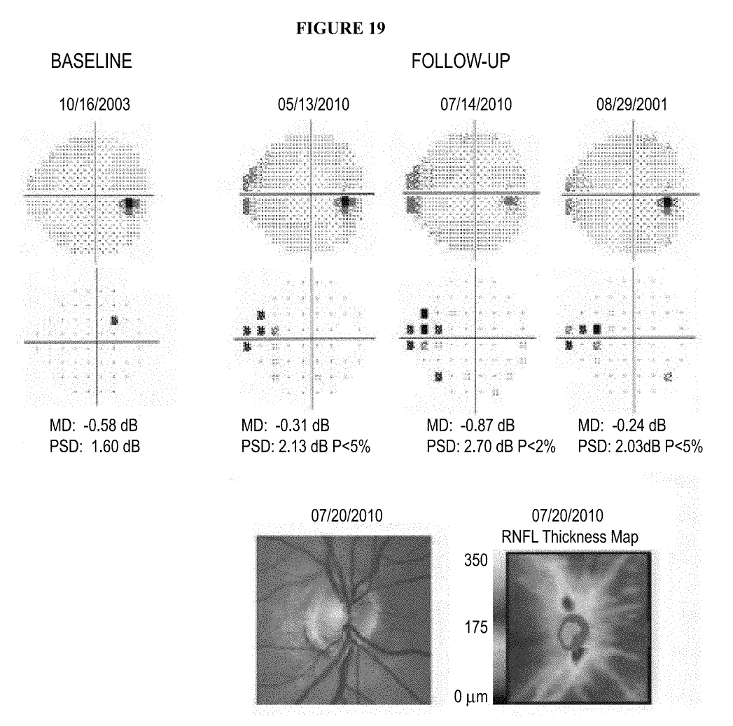

FIG. 19 shows an eye with an estimated RGC count of 800 369 at the time of development of the initial visual field defect, which corresponded to a 12% RGC loss compared with the healthy group. The optic disc photograph shows inferior neuroretinal rim thinning in agreement with inferior RNFL loss detected by SD-OCT. Average RNFL thickness was 80 μm. Visual fields show a more localized defect compared with the eye shown on FIG. 18, with an abnormal PSD but MD within normal limits.

### Discussion

In this study, empirical formulas were used to estimate RGC counts in suspect eyes converting to glaucoma at the time of the earliest development of visual field defects in comparison with a group of healthy eyes. Our results suggest that a substantial number of RGCs may be lost by the time early visual field changes are detectable on SAP. Eyes with early visual field defects in our study had an average estimated RGC count of 652 057 cells versus 910 584 cells in the healthy group with similar age. This translates into an estimated average RGC loss of 28.4% associated with early visual field defects. This number is remarkably similar to that found by Kerrigan-Baumrind et al.⁸⁰ in histologic studies of human eyes. The authors studied 17 postmortem eyes of 13 subjects with a well-documented history of glaucoma and compared the histologic RGC counts with those obtained from 17 postmortem eyes of 17 age-matched healthy controls. They found that the average RGC loss in eyes with PSD or corrected PSD with a P value less than 5% was 27.3%. These observations are also in agreement with other qualitative and quantitative clinical studies suggesting that substantial damage can occur to the optic nerve and RNFL before visual field defects are detectable on SAP.⁷¹⁻⁷⁹

To be able to estimate RGC losses associated with the earliest detectable visual field losses on SAP, we longitudinally followed a cohort of glaucoma suspects over time until they showed evidence of repeatable visual field defects. The criteria used to define visual field losses were those applied by the Ocular Hypertension Treatment Study^{74,95} and widely used in clinical practice, requiring confirmation of abnormalities in three consecutive visual fields. This greatly decreases the chance that the abnormalities seen on perimetry may represent just variability rather than true defects. The calculations of estimated RGC counts were performed at the time corresponding to the first abnormal visual field and therefore would reflect the amount of neural damage seen at the time of the first abnormality detected by perimetry in clinical practice. Because the eyes were observed during the transition period from normal to abnormal visual fields, this design provides a more robust determination of the point of earliest development of field losses than cross-sectional investigations.

Our method of estimating RGC counts relies on calculations of the number of RGCs estimated from data acquired by both SAP and OCT RNFL thickness evaluation. Empirical formulas for RGC count estimation from SAP and OCT were developed by Harwerth et al.⁸² By using normal monkeys and monkeys with laser-induced experimental glaucoma, they showed that SAP sensitivity values can provide good estimates of the amount of histologically measured RGC counts in the retina. These estimates agreed closely with those obtained from OCT RNFL thickness data. They showed a strong linear relationship between the number of RGC somas and axons obtained from functional and structural measures, respectively, when retinal eccentricity and appropriate measurement scales for neural and sensitivity losses were used. The linear relationship suggests that the lack of sensitivity of SAP for detection of early glaucomatous damage is most likely not the result of true structural changes occurring in the absence of functional losses, but is rather related to the logarithmic scale used for SAP sensitivity measurements, as well as the magnitude of change required to reach statistically significant levels of abnormality.^{72,96} The logarithmic scale compresses the range of losses in early stages of the disease while expanding the range in later stages. These findings could suggest that a simple linearization of SAP data could improve detection of early damage. However, although linearization of SAP measurements improves the structure and function relationship of population data, it generally does not improve the sensitivity to early losses in an individual patient.⁸³ Because SAP sensitivity thresholds are originally acquired using staircase procedures in decibel units, the compression of the range of losses in early stages of the disease caused by the logarithmic scale will still be present. Because of the weighting system for obtaining final RGC counts, our method relies more heavily on OCT data than SAP for estimation of early neural losses. However, it should be noted that there was still a significant contribution from SAP data in the RGC count estimates. This can be seen from the fact that estimated RGC counts performed significantly better than SD-OCT average thickness in discriminating glaucoma from healthy eyes, with ROC curve areas of 0.95 and 0.88, respectively, and higher sensitivities at fixed specificities. These results suggest that our proposed method for combining structural and functional data may perform better than isolated structural or functional tests for the detection of early glaucomatous damage. In addition, calculation of LRs for estimated RGC counts showed large effects on the probability of disease, giving further indication of the utility of this approach in clinical practice.⁹¹

In a previous investigation, we demonstrated that estimates of RGC counts obtained by the same method applied in the current study were able to detect preperimetric glaucomatous damage, that is, before the development of visual field defects.⁸³ Eyes with preperimetric damage had documented evidence of progressive glaucomatous damage on optic disc stereophotographs. These eyes had an average estimated loss of 17% of RGCs from age-expected RGC numbers. As expected, the average estimated percent RGC loss in eyes with visual field defects found in our study was greater than that of eyes with preperimetric damage. For eyes with moderate perimetric damage (average MD of −8.2 dB), the previously estimated average RGC loss was 52%, whereas for eyes with advanced damage (average MD of −17.4) it was 75%.⁸³ In another study, we showed that RGC counts performed better than isolated structural or functional parameters for detecting progressive glaucomatous damage over time.⁸⁴ The results of the present investigation combined with our previous studies suggest that our proposed method for estimating RGC counts could be a useful tool for the detection of glaucomatous damage throughout the spectrum of the disease.

Early detection and quantification of RGC losses in glaucoma may carry significant implications for the patient, even if they are not yet associated with detectable SAP losses. If substantial damage has already occurred by the time the disease is diagnosed, a relatively smaller number of RGCs will need to be lost before the number of cells reaches critical levels associated with disability from the disease. Although such critical levels presently cannot be ascertained for particular individuals, recent evidence suggests that a decrease in vision-related quality of life from glaucoma is observed sooner than previously anticipated.⁹⁷ Therefore, if treatment is initiated late in the course of the disease, a slower rate of change will have to be achieved to prevent the development of functional impairment than what would be necessary if treatment had been started earlier. Although it is generally possible to slow down the rate of disease progression and keep patients close to stability even if they have moderate or advanced damage,⁹⁸ this usually requires more aggressive interventions with a larger potential for side effects compared with what would be necessary if treatment had been started at an earlier stage. In 20% of the eyes with early visual field defects included in our study, the estimated RGC losses amounted to >40%, with an average RGC count of only 480 216 cells by the time the earliest visual field defect was detected on SAP. If we assume that functional impairment would occur with moderate to severe visual field damage, that is, with an RGC count of approximately 300 000 RGCs based on previous data,⁸³ these eyes would need to lose an additional 180 000 RGCs to go from early visual field defect to functional impairment, a lower number than what was lost before the development of early field defects. However, it is important to emphasize that the results of our study should not necessarily be taken as evidence that patients with optic nerve damage, but no apparent visual field loss, need to be treated. Although early treatment may be beneficial in many situations, decisions about treatment need to take into account several considerations, such as rate of disease progression, patient's life expectancy, risks of treatment, and patient's expectations about the disease and its treatment.

There was a large variation of the estimates of RGC losses in eyes with early visual field defects. This could be due to several reasons, such as variability of the tests used to estimate RGC counts, as well as the characteristics of the visual field defects detected by SAP. Because the Ocular Hypertension Treatment Study criterion used to detect visual field defects is essentially based on localized visual field losses or asymmetric damage on the Glaucoma Hemifield Test, it can potentially miss eyes with diffuse losses of sensitivity caused by diffuse neural losses in glaucoma. In fact, the eye illustrated in FIG. 18 shows extensive neural damage with an estimated average RGC loss of 43%, but only a relatively small localized visual field defect. However, there was evidence of diffuse visual field losses as measured by the MD of −2.14 dB (P<5%). On the other hand, the eye shown in FIG. 19 shows a more localized visual field defect without evidence of diffuse losses, and the estimated RGC damage was only 12%. This is in agreement with previous studies suggesting that pattern deviation analysis of SAP data may significantly underdiagnose glaucomatous eyes with diffuse losses of sensitivity.⁹⁹ Detection of eyes with diffuse loss of sensitivity is difficult because of the confounding effects of media opacities. This finding highlights the need for a combined approach of structural and functional evaluation for the detection of eyes with different patterns of glaucomatous damage.

### Study Limitations

We used empirically derived formulas to estimate the number of RGCs from SAP and OCT data, and our estimates of RGC counts were not based on direct histologic RGC counts in humans. The empirical formulas derived by Harwerth et al⁸² have been validated by histologic studies of monkeys that have a visual system almost indistinguishable from that of humans. The relationship between predicted RGC counts and histologically measured RGC numbers had an R² of 0.9, indicating an almost perfect predictive value. They have also been applied to multiple external cohorts in humans.⁸² There have been few to no histologic validations of measures, such as ganglion cell complex or even RNFL thickness as performed by OCT instruments. However, this carries little significance as long as one shows that these measurements have clinical relevance. Furthermore, our estimates agreed remarkably well with histologic studies of human glaucomatous eyes, as discussed earlier. Another limitation of our study is that we did not have longitudinal follow-up with SD-OCT over the same time course as SAP, which prevented us from obtaining estimates of RGC counts throughout the follow-up in glaucoma suspect eyes. However, it should be noted that even if longitudinal data on RGC counts were available, it would be impossible to determine the true individual amount of RGC loss from the disease because we currently have no way of determining when the glaucomatous damage started to occur. The design of our study addressed this limitation in the best possible way by comparing the estimates with an age-matched healthy population. We did not have follow-up data on the healthy eyes included in the control group, which would have allowed us to estimate age-related RGC losses and potentially better estimation of RGC losses in individual eyes. However, the patients with healthy eyes had an age similar to the patients in the glaucoma group; therefore, we still expect that our overall conclusions with regard to the average number of RGC losses would be correct.

In conclusion, glaucomatous eyes with the earliest detectable visual field losses on automated perimetry already show substantial losses of estimated RGC counts. Our proposed method to estimate RGC counts on the basis of a combination of structural and functional tests may allow detection and quantification of neural damage in these eyes with better diagnostic accuracy compared with standard parameters from imaging instruments.

Glaucoma is a leading cause of irreversible blindness and visual impairment in the world. The disease is characterized by progressive retinal ganglion cell (RGC) losses with associated characteristic structural changes at the level of the optic nerve and retinal nerve fiber layer (RNFL) which may lead to loss of visual function. The fundamental goal of glaucoma management is to prevent patients from developing visual impairment that is sufficient to produce disability in their daily lives and impair their health-related quality of life. However, due to the generally slowly progressive course of glaucoma, direct observation of disability endpoints is generally unfeasible for clinical trials testing new treatments for the disease.

Below limitations of endpoints traditionally used in clinical trials involving glaucoma patients are discussed. Developments in the field, such as the proposed use of structural measurements of the optic disc and RNFL for assessing progressive glaucomatous damage are also discussed, emphasizing their combined use along with functional measurements as a potential endpoint in the disease.

### Limitations of Current Endpoints

Although intraocular pressure (IOP) has traditionally been used as an endpoint in clinical trials, it is an imperfect surrogate for the clinically relevant outcomes of the disease. Many patients can progress despite low IOP levels and others remain stable despite having IOP measurements that are consistently high.¹⁰⁰⁻¹⁰² Further, IOP is not a suitable endpoint for clinical trials investigating certain treatment modalities for glaucoma, such as neuroprotective therapies. The use of visual fields as the sole endpoint in glaucoma trials is also potentially limited by the need for large samples, long-term follow-up and variability of results.¹⁰³ In the past two decades, a large bulk of evidence has accumulated with regard to the role of structural measurements of the optic disc and RNFL for diagnosing and detecting glaucoma progression. There is now substantial evidence that many patients can develop structural changes before appearance of detectable change in functional measures.^{104-109,110,111} Several studies have shown that optic disc and RNFL assessment by different imaging technologies such as optical coherence tomography (OCT), confocal scanning laser ophthalmoscopy and scanning laser polarimetry can provide objective and reliable assessment of rates of structural change in the disease. The use of structural measurements as surrogate endpoints in glaucoma clinical trials would have a number of advantages, including faster acquisition of a sufficient number of endpoints with reduction in sample size requirements, enabling shorter and less expensive trials.

### The Structure and Function Relationship in Glaucoma and Implications for Detection of Progression

Frequent disagreements are seen when structural and functional tests are used to monitor glaucoma patients for progression and this has led to confusion in the literature and among clinicians. These disagreements, however, are easily reconciled when one understands the nature of the structure and function relationship in the disease.¹¹² In fact, the very existence of disagreements is what makes it beneficial to employ combined approaches using both structure and function to increase the number of endpoints in clinical trials of the disease. The apparent disagreement between structural and functional measurements of the disease seem to be largely derived from the different algorithms and measurement scales as well as the different variability characteristics of the tests commonly used to assess structural and functional losses.^{112,113,114} In fact, Harwerth et al.¹¹³ demonstrated that structural and functional tests are in agreement as long as one uses appropriate measurement scales for neural and sensitivity losses and considers factors such as the effect of aging and eccentricity on estimates of neural losses. In a series of investigations, they demonstrated that estimates of RGC losses obtained from clinical standard automated perimetry (SAP) agreed closely with estimates of RGC losses obtained from RNFL assessment by OCT.¹¹³ The results of their model provided a common domain for expressing results of structural and functional tests, that is, the estimates of RGC losses, opening the possibility of combining these different tests to improve the reliability and accuracy of estimates of the amount of neural losses and develop a combined index for staging and detecting glaucomatous progression that could be used in clinical trials.

### Combining Structure and Function to Diagnose and Stage Glaucomatous Damage

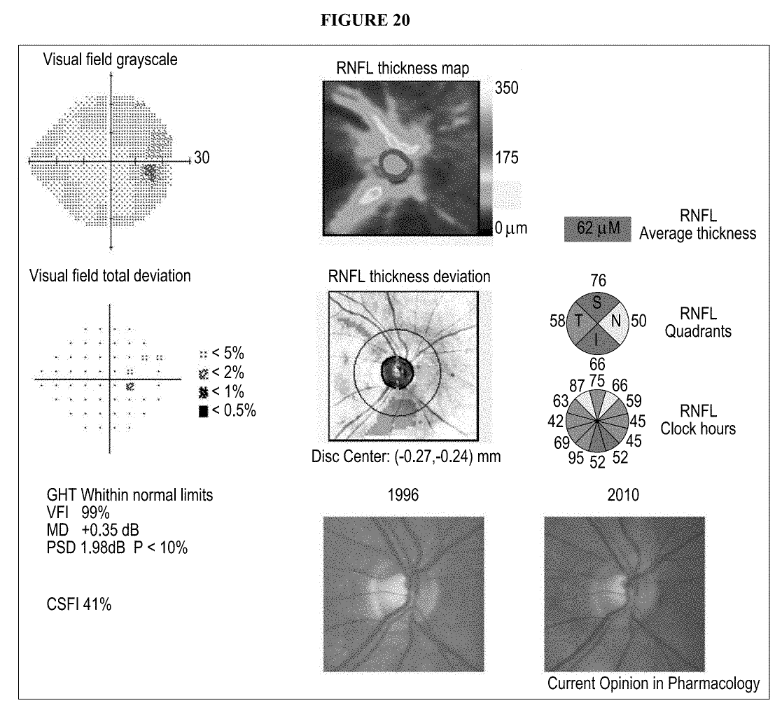

A combined structure and function index (CSFI) was described by Medeiros et al.¹¹⁵ with the purpose of merging the results of structural and functional tests into a single index that could be used for diagnosing, staging and detecting glaucomatous progression. The index uses estimates of RGC counts obtained by previously derived empirical formulas. The estimates of RGC counts are obtained from two sources: one structural, RNFL thickness assessed by OCT and one functional, standard automated perimetry. These estimates are then combined using a weighted average to provide a single estimate of the RGC count for a particular eye. For each eye, the CSFI represents the percent estimate of RGC loss compared with the age-expected number of RGCs (FIG. 20). By combining structural and functional tests into a single estimate of RGC loss, the index provides a very intuitive parameter to be used in clinical practice.

The CSFI has been shown to perform better than isolated structural and functional parameters for diagnosing and staging glaucomatous damage. Medeiros et al.¹¹⁵ evaluated the CSFI performance in a cross-sectional study involving 333 glaucomatous eyes and 165 healthy subjects. From the 333 glaucomatous eyes, 295 (89%) had perimetric glaucoma and 38 (11%) had preperimetric glaucoma. The mean CSFI, representing the mean estimated percent loss of RGCs, was 41% and 17% in the perimetric and preperimetric groups, respectively. The index had excellent diagnostic performance to detect glaucomatous eyes, with an area under the receiver operating characteristic (ROC) curve of 0.94. The index was also able to successfully detect eyes with preperimetric glaucoma, with ROC curve area of 0.85. This compared favorably to the usual parameters provided by SAP and spectral domain optical coherence tomography (SDOCT). FIG. 20 shows an example of an eye with preperimetric glaucomatous damage. This eye had evidence of documented progressive optic disc glaucomatous damage on stereophotographs. However, the visual field exam was still within normal limits. Results of the SDOCT exam showed superior and inferior RNFL thinning, with global average thickness of 62 μm. The CSFI for this eye was 41%, indicating an estimated 41% loss of RGCs compared to what would be expected for the age. This case illustrates the significant amount of RGC loss that can occur despite statistically normal visual fields.

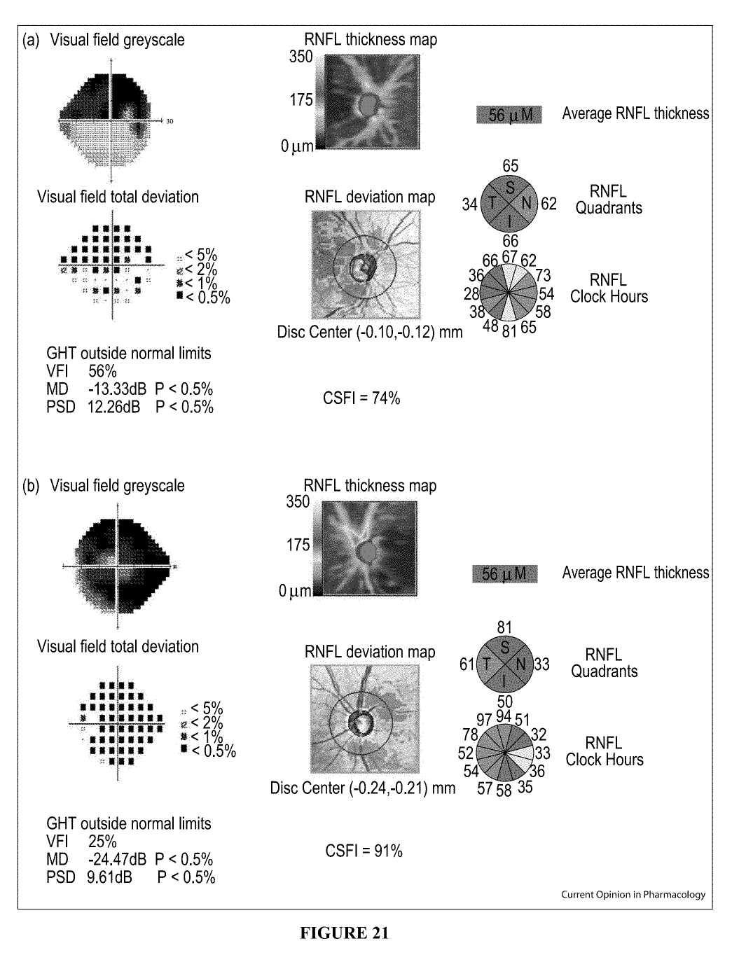

The CSFI was also shown to successfully stage different degrees of glaucomatous damage, which is an essential requirement for any method proposed to detect disease progression over time. To separate eyes with early from moderate visual field loss, the CSFI had ROC curve area of 0.94 compared to only 0.77 for SDOCT average thickness (P<0.001). Similarly, for separating moderate from advanced glaucomatous field loss, the ROC curve area of the CSFI was 0.96, which was again significantly better than that for average RNFL thickness (ROC area=0.70; P<0.001). FIG. 21 illustrates two eyes with different degrees of visual field loss (MDs of −13.3 dB and −24.5 dB) successfully discriminated by the CSFI but not by SDOCT results.

Some potential limitations of the CSFI are worth noting. The CSFI used empirically-derived formulas to estimate the number of RGCs from SAP and OCT data based on previous experimental studies in monkeys.¹¹³ Although estimates obtained from these formulas have been validated in multiple external cohorts including human data¹¹³, no studies have compared actual CSFI estimates with histological estimates of human glaucomatous eyes. It should be noted that there have been little to no histological validations of measures such as ganglion cell complex or even RNFL thickness as performed by OCT instruments. However, this carries little significance as long as one shows that these measurements have clinical relevance. Also, the original formula for estimating RGCs from OCT data was based on an older version of the OCT technology, time-domain OCT. It is possible that modifications might be necessary when using estimates based on SDOCT technology. Another potential limitation of the index is that the presence of media opacities could potentially affect SAP-derived estimates of RGCs and, therefore, calculations of the CSFI. This is a potential limitation of most visual field-based staging systems, as they usually base their classifications at least partly on values of the mean deviation index. However, by combining functional and structural measurements, the approach potentially reduces the effect of media opacities by relatively decreasing the influence of SAP-derived data on the final estimates of neuronal losses. Nevertheless, clinicians should be aware of the effect of media opacities when evaluating functional changes and quality of imaging test results in glaucoma patients.

### Combining Structure and Function to Assess Glaucoma Progression

As described above, frequent disagreements are seen when structural and functional tests are used to detect glaucomatous progression.¹¹² While SAP has relatively low sensitivity to identify progression at initial stages of the disease, structural assessment often performs poorly to identify change at advanced stages of damage.¹¹² Differences in performance of structural and functional tests have been recently investigated in a study comparing structural and functional measurements to estimates of RGC counts in glaucoma.¹¹² In that study, analysis of the relationship between visual field data and RGC counts indicated that, at early stages of the disease, significant losses of RGCs would correspond to relatively small changes in visual field parameters. This finding agrees with the large amount of evidence indicating that progressive optic disc or RNFL changes can frequently be seen before the appearance of statistically significant defects on SAP.^{100,102,104-106,111,114,116,117} Scaling of perimetric stimulus intensities has been incorporated into standard perimetric testing, where the stimulus intensities are scaled by a logarithmic transformation to decibel units of attenuation for both the intensity staircase procedure for threshold measurements as well as for the report of the final threshold intensity. Several investigators have suggested that such scaling may introduce an artifactual relationship between structural and functional measurements in glaucoma.^{114,116,118,119} The logarithmic scale would accentuate sensitivity changes in the visual field at low decibel values and minimize changes at high decibel levels, so that perimetry would be more suitable for detection of moderate to severe damage. On the contrary, analysis of the relationship between RNFL thickness and estimated RGC counts indicated that imaging instruments could be used to gauge information on rates of neural losses in early disease, when SAP evaluation can be misleading. However, at moderate to severe stages of the disease, evaluation of progressive damage with SDOCT becomes less helpful when the instrument reaches a floor level where it cannot detect further changes anymore.

Approaches combining structure and function can take advantage of the different performance of these tests according to the stage of damage in order to provide a reliable method for detecting change throughout the spectrum of the disease. It is important to emphasize that an optimal method for detecting glaucomatous progression should not only give an indication of whether or not the eye is changing over time, but also should estimate the rate of deterioration. Although most glaucoma patients will show some evidence of progression if followed long enough, the rate of deterioration can be highly variable among them.^{108,117,120-122} While most patients progress relatively slowly, others have aggressive disease with fast deterioration that can eventually result in blindness or substantial impairment unless appropriate interventions take place. The use of rates of change as the outcome variable may also result in decreased sample size requirements compared to the use of categorical classifications.

Estimates of RGC counts from a combination of structural and functional tests have been shown to be able to detect glaucomatous progression and estimate rates of disease deterioration.¹²³ In a longitudinal study of 213 eyes followed for an average of 4.5 years, 47 (22.1%) showed statistically significant rates of estimated RGC loss that were faster than the age-expected decline. The mean rate of estimated RGC loss in these eyes was −33 369 cells/year (range: −8332 cells/year to −80 636 cells/year). In addition, estimates of RGC losses detected a significantly larger number of progressing eyes compared to isolated measures of function and structure at the same specificity level.¹²³

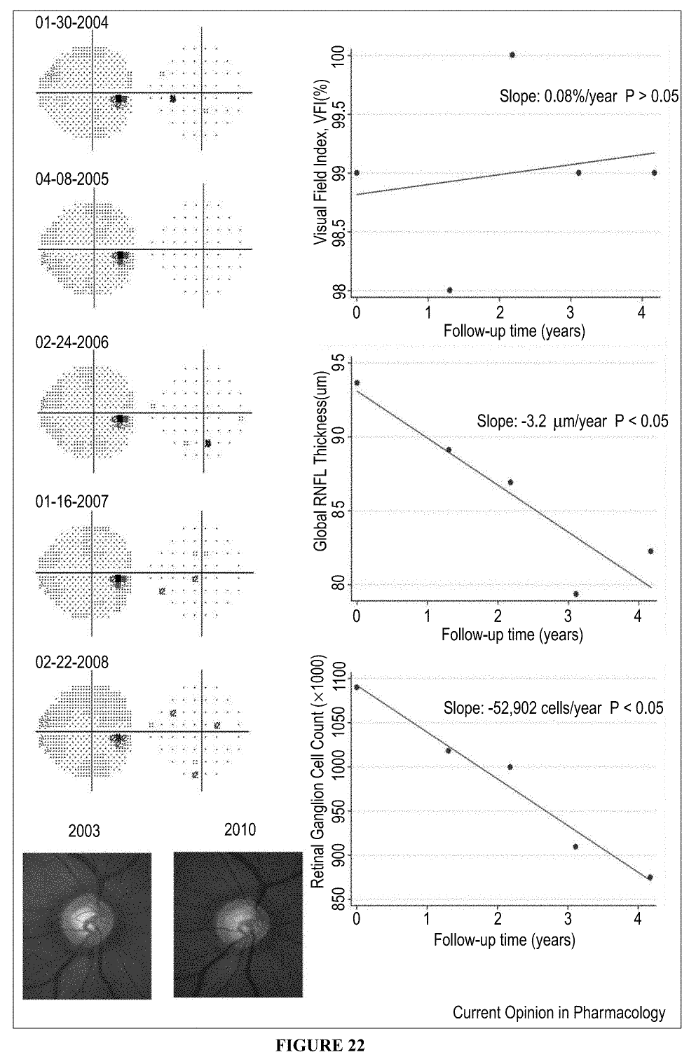

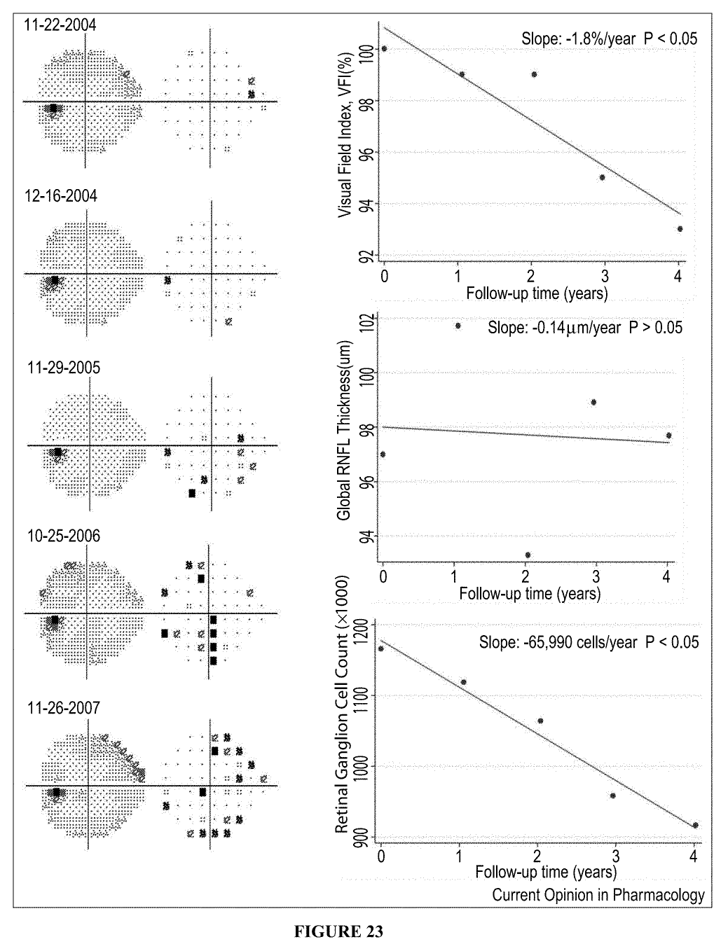

FIGS. 22 and 23 illustrate detection of glaucoma progression using estimated RGC counts. FIG. 22 shows an example of an eye with preperimetric damage that was detected as progressing by the rate of RGC loss and by the rate of global RNFL thickness change, but not by visual fields. By contrast, FIG. 23 shows an example of an eye that was detected as progressing by rate of RGC loss and by rate of visual field loss, but not by global RNFL thickness.

Limitations of the CSFI for staging the disease as described above would also apply for detection of glaucomatous progression over time, such as the possible influence of media opacities. In addition, original calculations of estimated RGC counts and CSFI have only considered global measurements. Due to the localized aspect of glaucomatous damage in many eyes, it is possible that a sectorial approach focusing on detection of localized RGC losses may improve detection of progressive damage.

Other approaches have been suggested to combine structural and functional tests to detect glaucomatous progression, including the use of Bayesian methodologies to allow combination of different tests.^{124,125} These approaches are effective in combining results of different tests to improve the estimates of rate of change and have the advantage of being capable of incorporating other covariates, such as demographic and clinical risk factors, to increase the accuracy and precision of the estimates.¹²⁶ However, Bayesian analyses have the disadvantage of not being intuitive for the majority of clinicians. Further studies are necessary to evaluate which approach provides the best use of resources for clinical trials in glaucoma.

### Conclusion

The use of combined approaches potentially provides a more effective means for detection of glaucoma progression and estimation of rates of change than structural or functional testing alone. Combined approaches also may provide more reliable identification of endpoints, potentially reducing sample size requirements for clinical trials investigating new therapies to prevent glaucomatous progression. A recently described approach estimating rates of retinal ganglion cell loss from a combination of structural and functional tests offers promise as a method for diagnosing, staging, detecting progression and estimating rates of glaucomatous deterioration. Its use in clinical trials may potentially overcome the limitations of currently available conventional parameters.

### Implementation

The system and method described herein can be implemented on various configurations of hardware and software. The system can be comprised of various modules, tools, and applications as discussed below. As can be appreciated by one of ordinary skill in the art, each of the modules may comprise various sub-routines, procedures, definitional statements and macros. Each of the modules are typically separately compiled and linked into a single executable program. Therefore, the following description of each of the modules is used for convenience to describe the functionality of a preferred system. Thus, the processes that are undergone by each of the modules may be arbitrarily redistributed to one of the other modules, combined together in a single module, or made available in, for example, a shareable dynamic link library. Depending on the embodiment, certain modules may be removed, merged together, or rearranged in order. Also depending on the embodiment, certain steps of the methods may be added, rearranged, combined, or removed.

The system modules, tools, and applications may be written in any programming language such as, for example, C, C++, C#, BASIC, Visual Basic, Pascal, Ada, Java, HTML, XML, or FORTRAN, and executed on an operating system, such as variants of Windows, Macintosh, UNIX, Linux, VxWorks, or other operating system. C, C++, C#, BASIC, Visual Basic, Pascal, Ada, Java, HTML, XML and FORTRAN are industry standard programming languages for which many commercial compilers can be used to create executable code.

### Definitions

The following provides a number of useful possible definitions of terms used in describing certain embodiments of the disclosed development.

A network may refer to a network or combination of networks spanning any geographical area, such as a local area network (LAN), wide area network (WAN), regional network, national network, and/or global network. The Internet is an example of a current global computer network. Those terms may refer to hardwire networks, wireless networks, or a combination of hardwire and wireless networks. Hardwire networks may include, for example, fiber optic lines, cable lines, ISDN lines, copper lines, etc. Wireless networks may include, for example, cellular systems, personal communications service (PCS) systems, satellite communication systems, packet radio systems, and mobile broadband systems. A cellular system may use, for example, code division multiple access (CDMA), time division multiple access (TDMA), personal digital phone (PDC), Global System Mobile (GSM), or frequency division multiple access (FDMA), among others. In addition, connectivity to the network may be, for example, via remote modem, Ethernet (IEEE 802.3), Token Ring (IEEE 802.5), Fiber Distributed Datalink Interface (FDDI) or Asynchronous Transfer Mode (ATM). As used herein, the network includes network variations such as the public Internet, a private network within the Internet, a secure network within the Internet, a private network, a public network, a value-added network, an intranet, and the like.

A website may refer to one or more interrelated web page files and other files and programs on one or more web servers. The files and programs are accessible over a computer network, such as the Internet, by sending a hypertext transfer protocol (HTTP or HTTPS [S-HTTP]) request specifying a uniform resource locator (URL) that identifies the location of one of the web page files, where the files and programs are owned, managed or authorized by a single business entity. Such files and programs can include, for example, hypertext markup language (HTML) files, common gateway interface (CGI) files, and Java applications. The web page files preferably include a home page file that corresponds to a home page of the website. The home page can serve as a gateway or access point to the remaining files and programs contained within the website. In one embodiment, all of the files and programs are located under, and accessible within, the same network domain as the home page file. Alternatively, the files and programs can be located and accessible through several different network domains.

A web page or electronic page may include that which is presented by a standard web browser in response to an HTTP request specifying the URL by which the web page file is identified. A web page can include, for example, text, images, sound, video, and animation.

A computer or computing device may be any processor controlled device. The computer or computing device may be a device that permits access to the Internet, including terminal devices, such as personal computers, workstations, servers, clients, mini-computers, main-frame computers, laptop computers, a network of individual computers, mobile computers, palm-top computers, hand-held computers, set top boxes for a television, other types of web-enabled televisions, interactive kiosks, personal digital assistants (PDAs), interactive or web-enabled wireless communications devices, mobile web browsers such as operating on a smartphone, or a combination thereof. The computers may further possess one or more input devices such as a keyboard, mouse, touch pad, joystick, pen-input-pad, and the like. The computers may also possess an output device, such as a visual display and an audio output. One or more of these computing devices may form a computing environment.

These computers may be uni-processor or multi-processor machines. Additionally, these computers may include an addressable storage medium or computer accessible medium, such as random access memory (RAM), an electronically erasable programmable read-only memory (EEPROM), programmable read-only memory (PROM), erasable programmable read-only memory (EPROM), hard disks, floppy disks, laser disk players, digital video devices, compact disks, video tapes, audio tapes, magnetic recording tracks, electronic networks, and other techniques to transmit or store electronic content such as, by way of example, programs and data. In one embodiment, the computers are equipped with a network communication device such as a network interface card, a modem, or other network connection device suitable for connecting to the communication network. Furthermore, the computers execute an appropriate operating system such as Linux, UNIX, any of the versions of Microsoft Windows, Apple MacOS, IBM OS/2 or other operating system. The appropriate operating system may include a communications protocol implementation that handles all incoming and outgoing message traffic passed over the network. In other embodiments, while the operating system may differ depending on the type of computer, the operating system will continue to provide the appropriate communications protocols to establish communication links with the network.

The computers may contain program logic, or other substrate configuration representing data and instructions, which cause the computer to operate in a specific and predefined manner, as described herein. In one embodiment, the program logic may be implemented as one or more object frameworks or modules. These modules may be configured to reside on the addressable storage medium and configured to execute on one or more processors. The modules include, but are not limited to, software or hardware components that perform certain tasks. Thus, a module may include, by way of example, components, such as, software components, object-oriented software components, class components and task components, processes, functions, attributes, procedures, subroutines, segments of program code, drivers, firmware, microcode, circuitry, data, databases, data structures, tables, arrays, and variables.

The various components of the system may communicate with each other and other components comprising the respective computers through mechanisms such as, by way of example, interprocess communication, remote procedure call, distributed object interfaces, and other various program interfaces. Furthermore, the functionality provided for in the components, modules, and databases may be combined into fewer components, modules, or databases or further separated into additional components, modules, or databases. Additionally, the components, modules, and databases may be implemented to execute on one or more computers. In another embodiment, some of the components, modules, and databases may be implemented to execute on one or more computers external to a website. In one instance, the website includes program logic, which enables the website to communicate with the externally implemented components, modules, and databases to perform the functions such as disclosed herein.

### Example Computing Environment

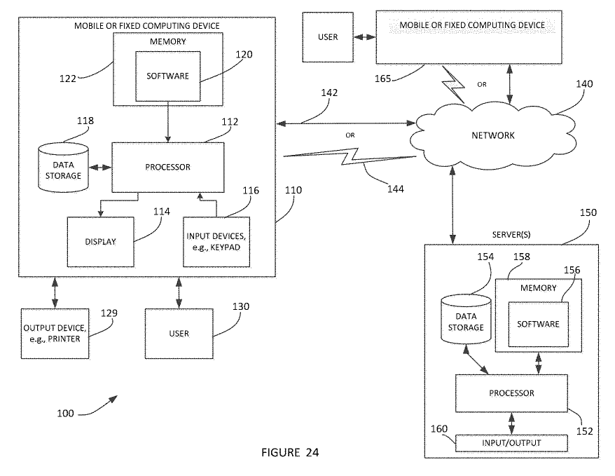

Certain embodiments of a system utilize a network as described in conjunction with FIG. 24. Certain embodiments are based on an example open system integrated architecture such as shown in FIG. 24. In FIG. 24, the example open system integrated architecture may be based on, for example, a user interface interacting with a local or remote data repository and a local or remote application running on a local or remote application server, such as an application server 150. FIG. 24 is a block diagram of an example system 100 that may be used to implement certain systems and methods described herein. The functionality provided for in the components and modules of computing system 100 may be combined into fewer components and modules or further separated into additional components and modules. Various other types of electronic devices communicating in a networked environment may also be used.

Referring to FIG. 24, an example configuration of components of an embodiment of the system 100 will now be described. A mobile or fixed computing device 110 is operated by a user 130. There may be other mobile or fixed computing devices such as a device 165 operated by other users. The computing device 110 can be a handheld computing device or other portable computing device such as a Palm, Pocket personal computer (PC), Linux based handheld, PDA, smartphone such as an iPhone® or Android™ based phone, a tablet computer such as an iPad® or Android based tablet, or a PC having a display. In other embodiments, the computing device can be any form of a network or Internet connected device, including but not limited to PCs, mobile devices, PDA, laptops, tablets, chips, keyboards, voice audio and video software, mouse, keypads, touch pads, track ball, microphones, videos, storage devices, network devices, databases, scanners, copiers, digital pens, image recognition software and device, screens and other forms of displays, netbooks and other forms of computer hardware. The computing device 110 in certain embodiments can operate in a stand-alone (independent) manner. In other embodiments, the computing device 110 is in communication with one or more servers 150 via a network 140, such as a local area network, a wide area network, or the Internet. The server(s) can include one or processors 152, memory 158, data storage 154 and system software 156 executed by the processor(s), and input or output devices 160. In certain embodiments, the data storage 154 stores one or more databases used by the system. The processor(s) 152 are in communication with the database(s) via a database interface, such as structured query language (SQL) or open database connectivity (ODBC). In certain embodiments, the data storage 154 is not included in server(s) 150, but is in data communication with the server(s) via the database interface. The connection from the computing device 110 to the network 140 can be a wireless or a satellite connection 144 or a wired or direct connection 142. In certain embodiments, the server(s) are part of a web site, such as a site on an intranet or the Internet.

When the computing device 110 is connected with the server(s) 150, the web site may optionally provide updates on new features. In another embodiment, the computing device runs software for the system and method described herein only when connected to the server(s) 150.

The computing device 110 can include a processor 112, memory 122, a display 114, and one or more input devices 116. The processor 112 can be in data communication with a data storage 118. In certain embodiments, the data storage 118 may store prior records of the user and/or other data or software. System software 120 can be executed by the processor 112. The system software 120 may include an application graphical user interface (GUI). The application GUI can include a database interface to the data storage 118 of the computing device. In certain embodiments, the software is loaded from the data storage 118. In embodiments where the computing device 110 communicates with a web site, the processor utilizes browser software in place of or in addition to the software 120. The network browser may be, for example, Microsoft Internet Explorer®, Apple Safari®, Mozilla Firefox®, Google Chrome™, browsers from Opera Software™, and so forth. An output device 129, such as a printer can be connected to the computing device 110.

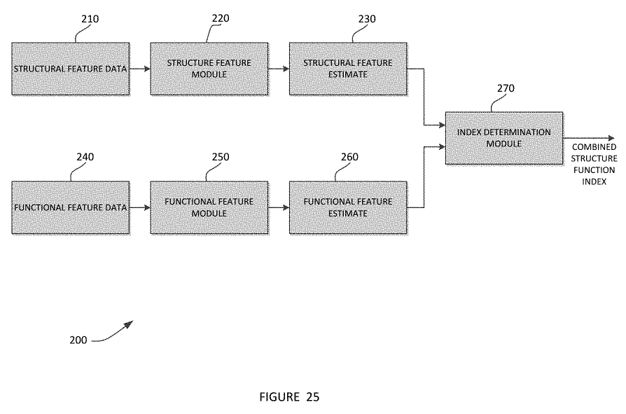

Referring to FIG. 25, an example top-level configuration 200 of modules will be described. Using this configuration, an index estimating a number of retinal ganglion cells in an eye can be determined. Computer implemented steps of the modules may be performed on the system 100 shown in FIG. 24. Depending on the embodiment, certain steps of the modules may be added, rearranged, combined, or removed.

Structural feature data 210, such as described above, is obtained and provided to a structure feature module 220. In certain embodiments, the structural feature data may be obtained from one of the data storages or databases described in conjunction with FIG. 24. The structure feature module 220, in certain embodiments, applies equations described above to estimate the number of RGC axons from RNFL thickness measurements obtained by OCT. The output of the structure feature module 220 is a structural feature estimate 230.

Functional feature data 240, such as previously described above, is obtained and provided to a functional feature module 250. In certain embodiments, the functional feature data may be obtained from one of the data storages or databases illustrated in FIG. 24. The functional feature module 250, in certain embodiments, applies equations described above to estimate the number of RGC somas in an area of the retina corresponding to a specific SAP test field location. The output of the functional feature module 250 is a functional feature estimate 260.

The structural feature estimate 230 and the functional feature estimate 260 are provided to an index determination module 270, which determines a weighted combination of the structural feature estimate and the functional feature estimate. The functional feature module 250, in certain embodiments, applies equations previously described above to determine a combined structure-function index.

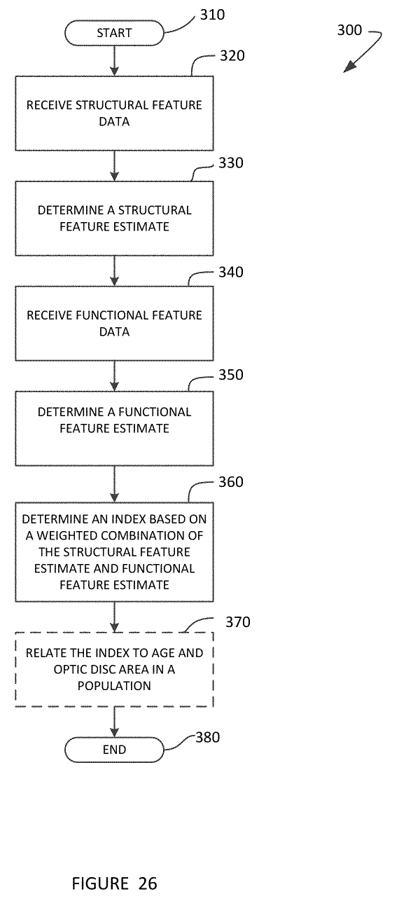

Referring to FIG. 26, an example flow 300 will be described. In flow 300, an index used to detect glaucoma or assess the progression of glaucoma is developed. Computer implemented steps of the flow may be performed on the system 100 shown in FIG. 24. Depending on the embodiment, certain steps of the flow may be added, rearranged, combined, or removed.

Beginning at a start state 310, flow 300 continues at state 320 where structural feature data, such as described above, is obtained. In certain embodiments, the structural feature data may be obtained from one of the data storages or databases described in conjunction with FIG. 24. Proceeding to state 330, a structural feature estimate is determined. In certain embodiments, the estimate is determined by applying equations described above to derive the total number of RGC axons from the global RNFL thickness measurement obtained by OCT.

Advancing to state 340, functional feature data, such as previously described above, is obtained. In certain embodiments, the functional feature data may be obtained from one of the data storages or databases described in conjunction with FIG. 24. Proceeding to state 350, a functional feature estimate is determined. In certain embodiments, the estimate is determined by applying equations described above to estimate the number of RGC somas in an area of the retina corresponding to a specific SAP test field location. Moving to a state 360, an index based on a weighted combination of the structural feature estimate and the functional feature estimate is determined. In certain embodiments, equations previously described above are applied to determine a combined structure-function index. Continuing at an optional state 370, a regression model is applied to relate the index to age and optic disc area in a population. Flow 300 completes at an end state 380.

### Clarifications Regarding Terminology

Those having skill in the art will further appreciate that the various illustrative logical blocks, modules, circuits, and process steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. One skilled in the art will recognize that a portion, or a part, may comprise something less than, or equal to, a whole. For example, a portion of a collection of pixels may refer to a sub-collection of those pixels.

The various illustrative logical blocks, modules, and circuits described in connection with the implementations disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or process described in connection with the implementations disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of non-transitory storage medium known in the art. An exemplary computer-readable storage medium is coupled to the processor such the processor can read information from, and write information to, the computer-readable storage medium. In the alternative, the storage medium may be integral to the processor. The processor and the storage medium may reside in an ASIC. The ASIC may reside in a user terminal, camera, or other device. In the alternative, the processor and the storage medium may reside as discrete components in a user terminal, camera, or other device.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

The disclosures of each of the following references and all references cited in the present application are incorporated herein by reference in their entireties.

### REFERENCES

1. Jonas J B, Budde W M, Panda-Jonas S. Ophthalmoscopic evaluation of the optic nerve head. Sury Ophthalmol 1999; 43:293-320.
2. Harwerth R S, Wheat J L, Fredette M J, Anderson D R. Linking structure and function in glaucoma. Prog Retin Eye Res 2010: 29:249-71.
3. Harwerth R S, Quigley H A. Visual field defects and retinal ganglion cell losses in patients with glaucoma. Arch Ophthalmol 2006: 124:853-9.
4. Quigley H A, Dunkelberger G R, Green W R. Retinal ganglion cell atrophy correlated with automated perimetry in human eyes with glaucoma. Am J Ophthalmol 1989: 107:453-64.
5. Medeiros F A, Alencar L M, Zangwill L M, et al. Prediction of functional loss in glaucoma from progressive optic disc damage. Arch Ophthalmol 2009: 127:1250-6.
6. Hood D C, Kardon R H. A framework for comparing structural and functional measures of glaucomatous damage. Prog Retin Eye Res 2007: 26:688-710.
7. Harwerth R S, Carter-Dawson L, Smith E L, 3rd, et al. Neural losses correlated with visual losses in clinical perimetry. Invest Ophthalmol Vis Sci 2004: 45:3152-60.
8. Kass M A, Heuer D K, Higginbotham E J, et al. The Ocular Hypertension Treatment Study: a randomized trial determines that topical ocular hypotensive medication delays or prevents the onset of primary open-angle glaucoma. Arch Ophthalmol 2002: 120:701-13; discussion 829-30.
9. Miglior S, Zeyen T, Pfeiffer N, et al. Results of the European Glaucoma Prevention Study. Ophthalmology 2005: 112:366-75.
10. Wollstein G, Schuman J S, Price L L, et al. Optical coherence tomography longitudinal evaluation of retinal nerve fiber layer thickness in glaucoma. Arch Ophthalmol 2005: 123:464-70.
11. Strouthidis N G, Scott A, Peter N M, Garway-Heath D F. Optic disc and visual field progression in ocular hypertensive subjects: detection rates, specificity, and agreement. Invest Ophthalmol Vis Sci 2006: 47:2904-10.
12. Leung C K, Cheung C Y, Weinreb R N, et al. Evaluation of retinal nerve fiber layer progression in glaucoma: a study on optical coherence tomography guided progression analysis. Invest Ophthalmol Vis Sci 2010: 51:217-22.
13. Medeiros F A, Alencar L M, Zangwill L M, et al. The Relationship between intraocular pressure and progressive retinal nerve fiber layer loss in glaucoma. Ophthalmology 2009: 116:1125-33 e1-3.
14. Alencar L M, Zangwill L M, Weinreb R N, et al. Agreement for detecting glaucoma progression with the GDx guided progression analysis, automated perimetry, and optic disc photography. Ophthalmology 2010: 117:462-70.
15. Medeiros F A, Alencar L M, Zangwill L M, et al. Detection of progressive retinal nerve fiber layer loss in glaucoma using scanning laser polarimetry with variable corneal compensation. Invest Ophthalmol Vis Sci 2009: 50:1675-81.
16. Medeiros F A, Leite M T, Zangwill L M, Weinreb R N. Combining Structural and Functional Measurements to Improve Detection of Glaucoma Progression using Bayesian Hierarchical Models. Invest Ophthalmol Vis Sci 2011: 52:5794-803.
17. Medeiros F A, Zangwill L M, Alencar L M, et al. Detection of glaucoma progression with stratus OCT retinal nerve fiber layer, optic nerve head, and macular thickness measurements. Invest Ophthalmol Vis Sci 2009: 50:5741-8.
18. Medeiros F A, Zangwill L M, Alencar L M, et al. Rates of progressive retinal nerve fiber layer loss in glaucoma measured by scanning laser polarimetry. Am J Ophthalmol 2010: 149:908-15.
19. Sample P A, Girkin C A, Zangwill L M, et al. The African Descent and Glaucoma Evaluation Study (ADAGES): design and baseline data. Arch Ophthalmol 2009: 127:1136-45.
20. Medeiros F A, Vizzeri G, Zangwill L M, et al. Comparison of retinal nerve fiber layer and optic disc imaging for diagnosing glaucoma in patients suspected of having the disease. Ophthalmology 2008: 115:1340-6.
21. Medeiros F A, Zangwill L M, Bowd C, et al. Use of progressive glaucomatous optic disk change as the reference standard for evaluation of diagnostic tests in glaucoma. Am J Ophthalmol 2005: 139:1010-8.
22. Racette L, Liebmann J M, Girkin C A, et al. African Descent and Glaucoma Evaluation Study (ADAGES): III. Ancestry differences in visual function in healthy eyes. Arch Ophthalmol 2010: 128:551-9.
23. Medeiros F A, Sample P A, Zangwill L M, et al. A statistical approach to the evaluation of covariate effects on the receiver operating characteristic curves of diagnostic tests in glaucoma. Invest Ophthalmol Vis Sci 2006: 47:2520-7.
24. Susanna R, Jr., Vessani R M. Staging glaucoma patient: why and how? Open Ophthalmol J 2009: 3:59-64.
25. Brusini P, Johnson C A. Staging functional damage in glaucoma: review of different classification methods. Sury Ophthalmol 2007: 52:156-79.
26. Brusini P. Staging the severity of disease in glaucoma. Arch Ophthalmol 2006: 124:1795-6; author reply 6.
27. Spaeth G L, Lopes J F, Junk A K, et al. Systems for staging the amount of optic nerve damage in glaucoma: a critical review and new material. Sury Ophthalmol 2006: 51:293-315.
28. Shields M B. Staging glaucoma for the twenty-first century: Sixth Chandler-Grant Lecture. J Glaucoma 1996: 5:340-4.
29. Mills R P, Budenz D L, Lee P P, et al. Categorizing the stage of glaucoma from pre-diagnosis to end-stage disease. Am J Ophthalmol 2006: 141:24-30.
30. Brusini P. GDx staging system: a new method for retinal nerve fiber layer damage classification. J Glaucoma 2011: 20:287-93.
31. Sihota R, Sony P, Gupta V, et al. Diagnostic capability of optical coherence tomography in evaluating the degree of glaucomatous retinal nerve fiber damage. Invest Ophthalmol Vis Sci 2006: 47:2006-10.
32. Leung C K, Cheung C Y, Weinreb R N, et al. Evaluation of retinal nerve fiber layer progression in glaucoma: a comparison between the fast and the regular retinal nerve fiber layer scans. Ophthalmology 2011: 118:763-7.
33. Garway-Heath D F, Caprioli J, Fitzke F W, Hitchings R A. Scaling the hill of vision: the physiological relationship between light sensitivity and ganglion cell numbers. Invest Ophthalmol Vis Sci 2000: 41:1774-82.
34. Medeiros F A, Weinreb R N, E R B, Rosen P N. Driving Simulation as a Performance-based Test of Visual Impairment in Glaucoma. J Glaucoma 2011.
35. Richman J, Lorenzana L L, Lankaranian D, et al. Importance of visual acuity and contrast sensitivity in patients with glaucoma. Arch Ophthalmol 2010: 128:1576-82.
36. Jonas J B, Budde W M, Panda-Jonas S. Ophthalmoscopic evaluation of the optic nerve head. Sury Ophthalmol. January-February 1999: 43(4):293-320.
37. Medeiros F A, Alencar L M, Zangwill L M, Bowd C, Sample P A, Weinreb R N. Prediction of functional loss in glaucoma from progressive optic disc damage. Arch Ophthalmol. October 2009: 127(10):1250-1256.
38. Hood D C, Kardon R H. A framework for comparing structural and functional measures of glaucomatous damage. Prog Retin Eye Res. November 2007: 26(6):688-710.
39. Harwerth R S, Carter-Dawson L, Smith E L, 3rd, Barnes G, Holt W F, Crawford M L. Neural losses correlated with visual losses in clinical perimetry. Invest Ophthalmol Vis Sci. September 2004: 45(9):3152-3160.
40. Kass M A, Heuer D K, Higginbotham E J, Johnson C A, Kellner J L, Miller J P, et al. The Ocular Hypertension Treatment Study: a randomized trial determines that topical ocular hypotensive medication delays or prevents the onset of primary open-angle glaucoma. Arch Ophthalmol. June 2002: 120(6):701-713; discussion 829-730.
41. Miglior S, Zeyen T, Pfeiffer N, Cunha-Vaz J, Toni V, Adamsons I. Results of the European Glaucoma Prevention Study. Ophthalmology. March 2005: 112(3):366-375.
42. Wollstein G, Schuman J S, Price L L, Aydin A, Stark P C, Hertzmark E, et al. Optical coherence tomography longitudinal evaluation of retinal nerve fiber layer thickness in glaucoma. Arch Ophthalmol. April 2005: 123(4):464-470.
43. Strouthidis N G, Scott A, Peter N M, Garway-Heath D F. Optic disc and visual field progression in ocular hypertensive subjects: detection rates, specificity, and agreement. Invest Ophthalmol Vis Sci. July 2006: 47(7):2904-2910.
44. Leung C K, Cheung C Y, Weinreb R N, Qiu K, Liu S, Li H, et al. Evaluation of retinal nerve fiber layer progression in glaucoma: a study on optical coherence tomography guided progression analysis. Invest Ophthalmol Vis Sci. January 2010: 51(1):217-222.
45. Medeiros F A, Alencar L M, Zangwill L M, Sample P A, Weinreb R N. The Relationship between intraocular pressure and progressive retinal nerve fiber layer loss in glaucoma. Ophthalmology. June 2009: 116(6):1125-1133 e1121-1123.
46. Artes P H, Chauhan B C. Longitudinal changes in the visual field and optic disc in glaucoma. Prog Retin Eye Res. May 2005: 24(3):333-354.
47. Harwerth R S, Wheat J L, Fredette M J, Anderson D R. Linking structure and function in glaucoma. Prog Retin Eye Res. July 2010: 29(4):249-271.
48. Sample P A, Girkin C A, Zangwill L M, Jain S, Racette L, Becerra L M, et al. The African Descent and Glaucoma Evaluation Study (ADAGES): design and baseline data. Arch Ophthalmol. September 2009: 127(9):1136-1145.
49. Racette L, Liebmann J M, Girkin C A, Zangwill L M, Jain S, Becerra L M, et al. African Descent and Glaucoma Evaluation Study (ADAGES): III. Ancestry differences in visual function in healthy eyes. Arch Ophthalmol. May 2010: 128(5):551-559.
50. Bengtsson B, Heijl A. False-negative responses in glaucoma perimetry: indicators of patient performance or test reliability? Invest Ophthalmol Vis Sci 2000: 41(8):2201-2204.
51. Medeiros F A, Zangwill L M, Bowd C, Vessani R M, Susanna R, Jr., Weinreb R N. Evaluation of retinal nerve fiber layer, optic nerve head, and macular thickness measurements for glaucoma detection using optical coherence tomography. Am J Ophthalmol. January 2005: 139(1):44-55.
52. Medeiros F A, Zangwill L M, Alencar L M, et al. Detection of glaucoma progression with Stratus OCT retinal nerve fiber layer, optic nerve head, and macular thickness measurements. Invest Ophthalmol Vis Sci 2009: 50(12):5741-5748.
53. Bengtsson B, Heijl A. A visual field index for calculation of glaucoma rate of progression. Am J Ophthalmol. February 2008: 145(2):343-353.
54. Williams R L. A note on robust variance estimation for cluster-correlated data. Biometrics 2000: 56(2):645- 646.
55. Alencar L M, Zangwill L M, Weinreb R N, Bowd C, Vizzeri G, Sample P A, et al. Agreement for detecting glaucoma progression with the GDx guided progression analysis, automated perimetry, and optic disc photography. Ophthalmology. March 2010: 117(3):462-470.
56. Fayers T, Strouthidis N G, Garway-Heath D F. Monitoring glaucomatous progression using a novel Heidelberg Retina Tomograph event analysis. Ophthalmology. November 2007: 114(11): 1973-1980.
57. Garway-Heath D F, Caprioli J, Fitzke F W, Hitchings R A. Scaling the hill of vision: the physiological relationship between light sensitivity and ganglion cell numbers. Invest Ophthalmol Vis Sci. June 2000: 41(7):1774-1782.
58. Medeiros F A, Lisboa R, Weinreb R N, Girkin C A, Liebmann J M, Zangwill L M. A combined index of structure and function for staging glaucomatous damage. Arch Ophthalmol 2012. Forthcoming.
59. Medeiros F A, Leite M T, Zangwill L M, Weinreb R N. Combining Structural and Functional Measurements to Improve Detection of Glaucoma Progression using Bayesian Hierarchical Models. Invest Ophthalmol Vis Sci. August 2011: 52(8):5794-5803.
60. Boland M V, Quigley H A. Evaluation of a combined index of optic nerve structure and function for glaucoma diagnosis. BMC Ophthalmol. 2011: 11:6.
61. Zhu H, Crabb D P, Fredette M J, Anderson D R, Garway-Heath D F. Quantifying discordance between structure and function measurements in the clinical assessment of glaucoma. Arch Ophthalmol. September 2011: 129(9):1167-1174.
62. Weinreb R N, Khaw P T. Primary open-angle glaucoma. Lancet. May 22 2004: 363(9422): 1711-1720.
63. Anderson D R, Drance S M, Schulzer M. Natural history of normal-tension glaucoma. Ophthalmology. February 2001: 108(2):247-253.
64. Heijl A, Bengtsson B, Hyman L, Leske M C. Natural history of open-angle glaucoma. Ophthalmology. December 2009: 116(12):2271-2276.
65. Medeiros F A, Zangwill L M, Alencar L M, Sample P A, Weinreb R N. Rates of progressive retinal nerve fiber layer loss in glaucoma measured by scanning laser polarimetry. Am J Ophthalmol. June 2010: 149(6):908-915.
66. Artes P H, O'Leary N, Hutchison D M, et al. Properties of the Statpac visual field index. Invest Ophthalmol Vis Sci 2011; 52(7):4030-4038.
67. Kerrigan-Baumrind L A, Quigley H A, Pease M E, Kerrigan D F, Mitchell R S. Number of ganglion cells in glaucoma eyes compared with threshold visual field tests in the same persons. Invest Ophthalmol Vis Sci 2000: 41(3):741-748.
68. Malik R, Swanson W H, Garway-Heath D F. Development and evaluation of a linear staircase strategy for the measurement of perimetric sensitivity. Vision Res 2006: 46(18):2956-2967.
69. Kim J S, Ishikawa H, Sung K R, Xu J, Wollstein G, Bilonick R A, et al. Retinal nerve fibre layer thickness measurement reproducibility improved with spectral domain optical coherence tomography. Br J Ophthalmol. August 2009: 93(8):1057-1063.
70. Quigley H A, Dunkelberger G R, Green W R. Retinal ganglion cell atrophy correlated with automated perimetry in human eyes with glaucoma. Am J Ophthalmol 1989: 107:453-64.
71. Medeiros F A, Alencar L M, Zangwill L M, et al. Prediction of functional loss in glaucoma from progressive optic disc damage. Arch Ophthalmol 2009: 127:1250-6.
72. Hood D C, Kardon R H. A framework for comparing structural and functional measures of glaucomatous damage. Prog Retin Eye Res 2007: 26:688-710.
73. Harwerth R S, Carter-Dawson L, Smith E L III, et al. Neural losses correlated with visual losses in clinical perimetry. Invest Ophthalmol Vis Sci 2004: 45:3152- 60.
74. Kass M A, Heuer D K, Higginbotham E J, et al, Ocular Hypertension Treatment Study Group. The Ocular Hypertension Treatment Study: a randomized trial determines that topical ocular hypotensive medication delays or prevents the onset of primary open-angle glaucoma. Arch Ophthalmol 2002: 120: 701-13; discussion 829-30.
75. European Glaucoma Prevention Study (EGPS) Group. Results of the European Glaucoma Prevention Study. Ophthalmology 2005: 112:366-75.
76. Wollstein G, Schuman J S, Price L L, et al. Optical coherence tomography longitudinal evaluation of retinal nerve fiber layer thickness in glaucoma. Arch Ophthalmol 2005: 123:464-70.
77. Strouthidis N G, Scott A, Peter N M, Garway-Heath D F. Optic disc and visual field progression in ocular hypertensive subjects: detection rates, specificity, and agreement. Invest Ophthalmol Vis Sci 2006: 47:2904-10.
78. Leung C K, Cheung C Y, Weinreb R N, et al. Evaluation of retinal nerve fiber layer progression in glaucoma: a study on optical coherence tomography guided progression analysis. Invest Ophthalmol Vis Sci 2010: 51:217-22.
79. Medeiros F A, Alencar L M, Zangwill L M, et al. The relationship between intraocular pressure and progressive retinal nerve fiber layer loss in glaucoma. Ophthalmology 2009: 116: 1125-33.
80. Kerrigan-Baumrind L A, Quigley H A, Pease M E, et al. Number of ganglion cells in glaucoma eyes compared with threshold visual field tests in the same persons. Invest Ophthalmol Vis Sci 2000: 41:741- 8.
81. Harwerth R S, Vilupuru A S, Rangaswamy N V, Smith E L III. The relationship between nerve fiber layer and perimetry measurements. Invest Ophthalmol Vis Sci 2007: 48:763-73.
82. Harwerth R S, Wheat J L, Fredette M J, Anderson D R. Linking structure and function in glaucoma. Prog Retin Eye Res 2010: 29:249-71.
83. Medeiros F A, Lisboa R, Weinreb R N, et al. A combined index of structure and function for staging glaucomatous damage. Arch Ophthalmol 2012: 130:1107-16.
84. Medeiros F A, Zangwill L M, Anderson D R, et al. Estimating the rate of retinal ganglion cell loss in glaucoma. Am J Ophthalmol 2012: 154:814-24.
85. Medeiros F A, Zangwill L M, Bowd C, et al. The structure and function relationship in glaucoma: implications for detection of progression and measurement of rates of change. Invest Ophthalmol Vis Sci 2012: 53:6939-46.
86. Sample P A, Girkin C A, Zangwill L M, et al, ADAGES Study Group. The African Descent and Glaucoma Evaluation Study (ADAGES): design and baseline data. Arch Ophthalmol 2009; 127:1136-45.
87. Racette L, Liebmann J M, Girkin C A, et al, ADAGES Group. African Descent and Glaucoma Evaluation Study (ADAGES): III. Ancestry differences in visual function in healthy eyes. Arch Ophthalmol 2010: 128:551-9.
88. Zhou X H, Obuchowski N A, McClish D K. Regression analysis of independent ROC data. In: Zhou X H, Obuchowski N A, McClish D K, eds. Statistical Methods in Diagnostic Medicine. New York: Wiley-Interscience; 2002:274-306.
89. Alonzo T A, Pepe M S. Distribution-free ROC analysis using binary regression techniques. Biostatistics 2002: 3:421-32.
90. Radack K L, Rouan G, Hedges J. The likelihood ratio. An improved measure for reporting and evaluating diagnostic test results. Arch Pathol Lab Med 1986: 110:689-93.
91. Jaeschke R, Guyatt G H, Sackett D L, Evidence-Based Medicine Working Group. Users' guides to the medical literature. III. How to use an article about a diagnostic test. B. What are the results and will they help me in caring for my patients? JAMA 1994: 271:703-7.
92. Fagan T J. Letter: Nomogram for Bayes theorem. N Engl J Med 1975: 293:257.
93. Medeiros F A, Zangwill L M, Bowd C, et al. Use of progressive glaucomatous optic disk change as the reference standard for evaluation of diagnostic tests in glaucoma. Am J Ophthalmol 2005: 139:1010-8.
94. Medeiros F A, Zangwill L M, Bowd C, Weinreb R N. Comparison of the GDx VCC scanning laser polarimeter, HRT II confocal scanning laser ophthalmoscope, and Stratus OCT optical coherence tomograph for the detection of glaucoma. Arch Ophthalmol 2004: 122:827-37.
95. Keltner J L, Johnson C A, Quigg J M, et al, Ocular Hypertension Treatment Study Group. Confirmation of visual field abnormalities in the Ocular Hypertension Treatment Study. Arch Ophthalmol 2000: 118:1187-94.
96. Garway-Heath D F, Caprioli J, Fitzke F W, Hitchings R A. Scaling the hill of vision: the physiological relationship between light sensitivity and ganglion cell numbers. Invest Ophthalmol Vis Sci 2000: 41:1774-82.
97. McKean-Cowdin R, Varma R, Wu J, et al. Severity of visual field loss and health-related quality of life. Am J Ophthalmol 2007: 143:1013-23.
98. AGIS Investigators. The Advanced Glaucoma Intervention Study (AGIS): 7. The relationship between control of intraocular pressure and visual field deterioration. Am J Ophthalmol 2000: 130:429-40.
99. Artes P H, Chauhan B C, Keltner J L, et al, Ocular Hypertension Treatment Study Group. Longitudinal and cross-sectional analyses of visual field progression in participants of the Ocular Hypertension Treatment Study. Arch Ophthalmol 2010: 128:1528-32.
100. Kass M A, Heuer D K, Higginbotham E J, Johnson C A, Keltner J L, Miller J P, Parrish R K, Wilson M R, Gordon M O: The ocular hypertension treatment study: a randomized trial determines that topical ocular hypotensive medication delays or prevents the onset of primary open-angle glaucoma. Arch. Ophthalmol. 2002, 120:701-713 discussion 829-30.
101. Drance S M: The collaborative normal-tension glaucoma study and some of its lessons. Can. J. Ophthalmol. 1999, 34:1-6.
102. Miglior S, Zeyen T, Pfeiffer N, Cunha-Vaz J, Toni V, Adamsons I: Results of the European glaucoma prevention study. Ophthalmology 2005, 112:366-375.
103. Katz J, Congdon N, Friedman D S: Methodological variations in estimating apparent progressive visual field loss in clinical trials of glaucoma treatment. Arch. Ophthalmol. 1999, 117:1137-1142.
104. Leung C K, Cheung C Y L, Weinreb R N, Qiu K, Liu S, Li H, Xu G, Fan N, Pang C P, Tse K K, Lam D S C: Evaluation of retinal nerve fiber layer progression in glaucoma: a study on optical coherence tomography guided progression analysis. Invest. Ophthalmol. Vis. Sci. 2010, 51:217-222.
105. Medeiros F A, Alencar L M, Zangwill L M, Bowd C, Sample P A, Weinreb R N: Prediction of functional loss in glaucoma from progressive optic disc damage. Arch. Ophthalmol. 2009, 127:1250-1256.
106. Strouthidis N G, Scott A, Peter N M, Garway-Heath D F: Optic disc and visual field progression in ocular hypertensive subjects: detection rates, specificity, and agreement. Invest. Ophthalmol. Vis. Sci. 2006, 47:2904-2910.
107. Medeiros F A, Zangwill L M, Alencar L M, Bowd C, Sample P A, Susanna R Jr, Weinreb R N: Detection of glaucoma progression with stratus OCT retinal nerve fiber layer, optic nerve head, and macular thickness measurements. Invest. Ophthalmol. Vis. Sci. 2009, 50:5741-5748.
108. Medeiros F A, Zangwill L M, Alencar L M, Sample P A, Weinreb R N: Rates of progressive retinal nerve fiber layer loss in glaucoma measured by scanning laser polarimetry. Am. J. Ophthalmol. 2010, 149:908-915.
109. Medeiros F A, Zangwill L M, Bowd C, Sample P A, Weinreb R N: Use of progressive glaucomatous optic disk change as the reference standard for evaluation of diagnostic tests in glaucoma. Am. J. Ophthalmol. 2005, 139:1010-1018.
110. Leung C K, Chiu V, Weinreb R N, Liu S, Ye C, Lai G, Liu N, Pang C P, Tse K K, Lam D S C: Evaluation of retinal nerve fiber layer progression in glaucoma: a comparison between spectraldomain and time-domain optical coherence tomography. Ophthalmology 2011, 118:1558-1562.
111. Wollstein G, Schuman J S, Price L L, Aydin A, Stark P C, Hertzmark E, Lai E, Ishikawa H, Mattox C, Fujimoto J G: Optical coherence tomography longitudinal evaluation of retinal nerve fiber layer thickness in glaucoma. Arch. Ophthalmol. 2005, 123:464-470.
112. Medeiros F A, Zangwill L M, Bowd C, Mansouri K, Weinreb R N: The structure and function relationship in glaucoma: implications for detection of progression and measurement of rates of change. Invest. Ophthalmol. Vis. Sci. 2012, 53:6939-6946.
113. Harwerth R S, Wheat J L, Fredette M J, Anderson D R: Linking structure and function in glaucoma. Prog. Retin. Eye Res. 2010, 29:249-271.
114. Hood D C, Kardon R H: A framework for comparing structural and functional measures of glaucomatous damage. Prog. Retin. Eye Res. 2007, 26:688-710.
115. Medeiros F A, Lisboa R, Weinreb R N, Girkin C A, Liebmann J M, Zangwill L M: A combined index of structure and function for staging glaucomatous damage. Arch. Ophthalmol. 2012, 130:E1-E10.
116. Harwerth R S, Carter-Dawson L, Smith E L 3rd, Barnes G, Holt W F, Crawford M L: Neural losses correlated with visual losses in clinical perimetry. Invest. Ophthalmol. Vis. Sci. 2004, 45:3152-3160.
117. Medeiros F A, Alencar L M, Zangwill L M, Sample P A, Weinreb R N: The relationship between intraocular pressure and progressive retinal nerve fiber layer loss in glaucoma. Ophthalmology 2009, 116:1125-1133 e1-3.
118. Garway-Heath D F, Caprioli J, Fitzke F W, Hitchings R A: Scaling the hill of vision: the physiological relationship between light sensitivity and ganglion cell numbers. Invest. Ophthalmol. Vis. Sci. 2000, 41:1774-1782.
119. Swanson W H, Felius J, Pan F: Perimetric defects and ganglion cell damage: interpreting linear relations using a two-stage neural model. Invest. Ophthalmol. Vis. Sci. 2004, 45:466-472.
120. Weinreb R N, Khaw P T: Primary open-angle glaucoma. Lancet 2004, 363:1711-1720.
121. Anderson D R, Drance S M, Schulzer M: Natural history of normaltension glaucoma. Ophthalmology 2001, 108:247-253.
122. Heijl A, Bengtsson B, Hyman L, Leske M C: Natural history of open-angle glaucoma. Ophthalmology 2009, 116:2271-2276.
123. Medeiros F A, Zangwill L M, Anderson D R, Liebmann J M, Girkin C A, Harwerth R S, Fredette M J, Weinreb R N: Estimating the rate of retinal ganglion cell loss in glaucoma. Am. J. Ophthalmol. 2012, 154:814-824.
124. Medeiros F A, Leite M T, Zangwill L M, Weinreb R N: Combining structural and functional measurements to improve detection of glaucoma progression using Bayesian hierarchical models. Invest. Ophthalmol. Vis. Sci. 2011, 52:5794-5803.
125. Russell R A, Malik R, Chauhan B C, Crabb D P, Garway-Heath D F: Improved estimates of visual field progression using Bayesian linear regression to integrate structural information in patients with ocular hypertension. Invest. Ophthalmol. Vis. Sci. 2012, 53:2760-2769.
126. Medeiros F A, Zangwill L M, Mansouri K, Lisboa R, Tafreshi A, Weinreb R N: Incorporating risk factors to improve the assessment of rates of glaucomatous progression. Invest. Ophthalmol. Vis. Sci. 2012, 53:2199-2207.

1-52. (canceled)

53. A method of evaluating a number of retinal ganglion cells (RGC) in an eye of a patient, the method comprising:
collecting structural measurements of the retina of a patient using an optical coherence tomography (OCT) device, said OCT measurements corresponding to scattering intensity as a function of depth in the eye;
collecting visual field defect measurements on the eye of a patient using a perimetry device;
determining a first estimate of the number of retinal ganglion cells based on the OCT measurements;
determining a second estimate of the number of retinal ganglion cells based on the perimetry measurements;
determining an index value corresponding to the number of retinal ganglion cells in the eye of the patient based on a weighted combination of the first estimate and the second estimate, wherein the weighting is based on the severity of the disease; and
storing or displaying the index value or a further analysis thereof.

54-66. (canceled)

67. The method of claim 53, wherein collecting structural measurements comprises estimating the number of RGC axons from RNFL thickness measurements obtained by optical coherence tomography.

68-78. (canceled)

79. The method of claim 53, wherein when determining the index value, more weight is given to the OCT measurements in the early stages of disease while more weight is given to the perimetry measurements in later stages of disease.

80. The method of claim 53, additionally comprising repeating the steps recited in claim 53 for the patient at a subsequent time, and determining a rate of glaucomatous deterioration and/or an extent of glaucomatous progression based on a change in the index value over time.

81. The method of claim 53, additionally comprising determining the second estimate by evaluating a linear function relating ganglion cell quantity in decibels to a visual field sensitivity in decibels at a given eccentricity, and by further adding estimates from all eccentricities to obtain a total ganglion cell count.

82. The method of claim 67, additionally comprising determining the first estimate by estimating a number of RGC axons from RNFL thickness measurements based on at least an effect of age and disease severity on an axonal density.

83. The method of claim 53, additionally comprising determining the second estimate using at least a portion of the perimetry measurements by applying at least the following equations:
m=[0.054*(ec*1.32)]+0.9
b=[−1.5*(ec*1.32)]−14.8
gc={[(s−1)−b]/m}+4.7
SAPrgc=Σ10̂(gc*0.1)
wherein m and b represent a slope and intercept, respectively, of a linear function relating ganglion cell quantity, gc, in decibels to the visual sensitivity, s, in decibels at a given eccentricity, ec.

84. The method of claim 67, additionally comprising determining the first estimate using at least a portion of the OCT measurements by applying at least the following equations:
d=(−0.007*age)+1.4
c=(−0.26*MD)+0.12
a=average RNFL thickness*10870*d
OCTrgc=10̂[(log(a)*10−c)*0.1]
wherein d corresponds to an axonal density, c is a correction factor for the severity of disease, age is an age of the patient, and MD comprises a mean deviation.

85. The method of claim 67, additionally comprising determining the second estimate using at least a portion of the perimetry measurements by applying at least the following equations:
m=[0.054*(ec*1.32)]+0.9
b=[−1.5*(ec*1.32)]−14.8
gc={[(s−1)−b]/m}+4.7
SAPrgc=Σ10̂(gc*0.1)
wherein m and b represent a slope and intercept, respectively, of a linear function relating ganglion cell quantity, gc, in decibels to a visual sensitivity, s, in decibels at a given eccentricity, ec;
determining the first estimate using at least a portion of the OCT measurements by applying at least the following equations:
d=(−0.007*age)+1.4
c=(−0.26*MD)+0.12
a=average RNFL thickness*10870*d
OCTrgc=10̂[(log(a)*10−c)*0.1]
wherein d corresponds to an axonal density, c is a correction factor for the severity of disease, age is an age of the patient and MD comprises a mean deviation; and
determining the weighted combination of the first estimate and the second estimate by applying at least the following formula:
wrgc=(1+MD/30)*OCTrgc+(−MD/30)*SAPrgc
wherein wrgc comprises at least a portion of the index.

86. The method of claim 53, additionally comprising implementing a regression model to relate the index to age and optic disc area in a population.

87. The method of claim 86, additionally comprising determining an expected retinal ganglion cell number according to age and optic disc area, and determining a combined structure-function index as:
[(expected retinal ganglion cell number−weighted combination of the first estimate and the second estimate)/(expected retinal ganglion cell number)]*100.

88. The method of claim 87, wherein the weighted combination of the first estimate and the second estimate comprises:
(1+MD/30)*first estimate+(−MD/30)*second estimate, wherein MD comprises a mean deviation.

89. The method of claim 87, wherein the combined structure-function index estimates the percent retinal ganglion cell loss.

90. The method of claim 87, wherein the combined structure-function index is used to diagnose or stage glaucoma.

91. The method of claim 86, wherein the regression model utilizes one of: a linear regression, an ordinary least squares (OLS) linear regression, and a locally weighted scatterplot smoothing.

92. The method of claim 53, wherein the index is determined using a device selected from the group consisting of a wired device, a wireless device, a plug-in device, a computer, an external input device and a combination of any of the foregoing devices.
